(19) United States
(12) Patent Application Publication
(10) Pub. No.: US 2023/0151080 A1
(43) Pub. Date: May 18, 2023

(54) METHODS FOR IDENTIFYING CORONAVIRUS CROSS-REACTING ANTIBODIES

(71) Applicant: LEYDEN LABORATORIES B.V., Amsterdam (NL)

(72) Inventors: Jaap Goudsmit, Amsterdam (NL); Marit Johanna Van Gils, Amsterdam (NL); Rogier Willem Sanders, Amsterdam (NL)

(21) Appl. No.: 18/004,719

(22) PCT Filed: Jul. 9, 2021
(86) PCT No.: PCT/NL2021/050435

§ 371 Date: Jan. 9, 2023

(30) Foreign Application Priority Data
Jul. 10, 2020 (NL) 2026043

(51) Int. Cl. C07K 16/10; A61P 31/14; G01N 33/50

(52) U.S. Cl. CPC C07K 16/10; A61P 31/14; G01N 33/5011

(57) ABSTRACT

The disclosure provides methods for identifying coronavirus cross-reacting antibodies. Such antibodies bind to at least part of the S2 ectodomain of the S protein of at least one common human coronavirus selected from HCoV-NL63, HCoV-OC43, HCoV-229E and HCoV-HKU1 and bind to at least part of the S2 ectodomain of the S protein of at least one highly pathogenic human coronavirus selected from SARS-CoV-1, MERS-CoV and SARS-CoV-2. Antibodies identified by the methods described herein are particularly useful for treating or preventing coronaviral infections, in particular against highly pathogenic coronaviruses such as SARS-CoV-1, MERS-CoV and/or SARS-CoV-2 as well as cross-species transmission of typically non-human coronaviruses.

### CROSS-REFERENCE TO RELATED APPLICATIONS

This application is a national phase entry under 35 U.S.C. § 371 of International Patent Application PCT/NL2021/050435, filed Jul. 9, 2021, designating the United States of America and published in English as International Patent Publication WO 2022/010353 on Jan. 13, 2022, which claims the benefit under Article 8 of the Patent Cooperation Treaty to Netherlands Patent Application Serial No. 2026043, filed Jul. 10, 2020, the entireties of which are hereby incorporated by reference.

### FIELD OF THE INVENTION

The disclosure provides methods for identifying coronavirus cross-reacting antibodies. Such antibodies bind to at least part of the S2 ectodomain of the S protein of at least one common human coronavirus selected from HCoV-NL63, HCoV-OC43, HCoV-229E and HCoV-HKU1 and bind to at least part of the S2 ectodomain of the S protein of at least one highly pathogenic human coronavirus selected from SARS-CoV-1, MERS-CoV and SARS-CoV-2. Antibodies identified by the methods described herein are particularly useful for treating or preventing coronaviral infections, in particular against highly pathogenic coronaviruses such as SARS-CoV-1, MERS-CoV and/or SARS-CoV-2 as well as cross-species transmission of typically non-human coronaviruses.

### BACKGROUND OF THE INVENTION

Coronaviruses are enveloped RNA viruses that can infect mammals and birds. Alphacoronaviruses and betacoronaviruses infect mammals (e.g., bovine coronavirus (BCoV); canine coronavirus (CCoV), feline coronavirus (FCoV), and human coronavirus (HCoV), while gammacoronaviruses and deltacoronaviruses infect generally infect birds. Most coronaviruses infect only one host species. However, cross-species transmission can also occur and is a significant cause of disease emergence in humans (i.e., zoonosis).

Coronaviruses encode a number of viral proteins including the spike protein, membrane protein, envelope protein and the nucleocapsid protein. The spike protein (S protein) is a large type I transmembrane, class I fusion protein. The ectodomain of the S protein contains an S1 domain and an S2 domain. The N-terminal S1 domain comprises receptor binding domains (RBD) and is responsible for receptor binding. The S1 domain, in particular the S1 RBD, has been a target site of a number of antibodies and vaccines developed against specific coronaviruses. The C-terminal S2 ectodomain is responsible for fusion and comprises an UH domain (upstream helix), a fusion peptide, two heptad repeats (HR1 and HR2), a central helix, and a beta hairpin. These regions and exemplary sequences of such regions are known in the art and sequence alignments of coronaviruses have been reported previously (see, e.g., Walls et al. Nature 2016 531:114-117 in particular Extended Data FIG. 9).

Seven strains of coronaviruses are known to infect humans. Infection by four of the human coronaviruses, i.e., HCoVs-229E, OC43, NL63, and HKU1 infections typically result in mild to severe upper and lower respiratory tract disease. These viruses account of approximately 15% of common colds. Infection by three of the human coronaviruses, i.e., Middle East respiratory syndrome-related coronavirus (MERS-CoV), Severe acute respiratory syndrome coronavirus (SARS-CoV), and Severe acute respiratory syndrome coronavirus 2 (SARS-CoV-2); can lead to severe symptoms as well as death. Humans likely acquired MERS-CoV from dromedary camels; SARS-CoV from bats; and bats may also have been the reservoir host for SARS-CoV-2.

Viral receptor engagement and viral fusion are essential for virus entry in a host cell. The spike protein of coronaviruses binds to different targets to mediate infectivity. SARS-CoV-2, SARS-CoV-1 and NL63 bind to ACE2, OC43 and HKU1 bind to 9-O-acetylated sialic acid, MERS-CoV binds to DPP4 and sialic acid, and 229E binds to APN. A further differentiating factor between these viruses is the presence or absence in the viral spike protein S of a human furin cleavage site. It is present in the S protein of SARS-CoV-2, OC43, HKU1, and MERS-CoV, but absent from the S protein of NL43, 229E and SARS-CoV.

SARS-CoV-2 is also referred to as COVID-19 virus (i.e., the novel coronavirus that causes coronavirus disease 2019). The Covid-19 pandemic has resulted in an enormous health crisis for which novel solutions are urgently needed to prevent, ameroliate or cure this infection. One object on the present disclosure is to provide methods and compositions for increasing immunity against SARS-CoV-2, as well as other pathogenic coronaviruses.

The major risk group for severe COVID-19 resulting in hospitalization peaks at ages between 70 and 80 and COVID-19 mortality peaks between 80 and 90 in countries like the Netherlands. This group has increasing numbers of comorbidities of which the majority is non-communicable. COVID-19 is thus an emergent disease of the aging, like pneumococcal pneumonia, severe influenza, shingles and pertussis (Santesmasses D et al. COVID-19 is an emergent disease of the aging, MedRxiv 2020). The same groups suffer from waning immunity and poor responsiveness to vaccines targeted at this age group. This remains a major stumbling block for vaccine efficacy in the elderly.

### SUMMARY OF THE INVENTION

While not wishing to be bound by theory, the disclosure proposes that infection, in particular simultaneous or sequential infection, by “common” HCoVs (e.g., HCoVs-229E, OC43, NL63, and HKU1) induces cross-reactive B-cells against heterologous virus strains, such as pathogenic HCoVs. The methods disclosed herein harness these cross-reactive B-cells together with in vivo models in order to identify antibodies that provide in vivo pan-corona cross-protection. Such methods differ from conventional studies which select antibody candidates based on, e.g., in vitro neutralization activity studies or from convalescent COVID-19 patients.

Antibodies identified from the methods described herein are especially useful for protecting subjects at risk of developing severe and potentially life-threating infections, including severe acute respiratory syndrome coronavirus 2 (SARS-CoV-2). Such antibodies may also be useful as a first line of protection against cross-species transmission of coronaviruses.

The disclosure provides the following preferred embodiments. However, the invention is not limited to these embodiments.

In one aspect a method is provided for identifying a coronavirus cross-reacting antibody, said method comprising

a) providing plasma samples from one or more human subjects, preferably at least 45 years or older, said samples collected, independently, at a time point (X),

b) optionally and preferably, identifying subjects having plasma samples with immunoglobulins that bind to at least two, preferably at least four, human coronaviruses (HCoV), wherein the HCoV is selected from HCoV-NL63, HCoV-OC43, HCoV-229E and HCoV-HKU1;

c) providing PBMC samples from said identified subjects, wherein the PBMC samples are collected at time point (X) or later and comprise B-cells selected from memory B-cells, plasma cells, and plasmablasts;

d) screening antibodies, or antigen-binding fragments thereof, encoded by the B-cells of c) for binding to at least part of the S2 ectodomain of the S (spike) protein from at least two, preferably at least four, different coronaviruses; preferably screening antibodies, or antigen-binding fragments thereof, for binding to at least part of the fusion peptide, the HR1 heptad repeat, or the HR2 heptad repeat of the S protein from at least two, preferably at least four, different coronaviruses

e) selecting antibodies, or antigen-binding fragments thereof, that bind to at least part of the S2 ectodomain of the S protein of at least one common human coronavirus selected from HCoV-NL63, HCoV-OC43, HCoV-229E and HCoV-HKU1 and that bind to at least part of the S2 ectodomain of the S protein of at least one highly pathogenic human coronavirus selected from SARS-CoV-1, MERS-CoV and SARS-CoV-2; preferably wherein said selected antibody or antigen-binding fragment thereof also binds to at least part of the S2 ectodomain of the S protein of an animal coronavirus; preferably selecting antibodies, or antigen-binding fragments thereof, that bind to at least part of the S2 domain of the S protein of HCoV-NL63, HCoV-OC43, HCoV-229E and HCoV-HKU1; preferably selecting antibodies, or antigen-binding fragments thereof, that bind to at least part of S2 domain of the S protein of SARS-CoV-1, MERS-CoV and SARS-CoV2;

f) selecting antibodies or antigen-binding fragments thereof from e) that inhibit viral fusion, infection, and/or replication of at least one common human coronavirus selected from HCoV-NL63, HCoV-OC43, HCoV-229E and HCoV-HKU1 and that inhibit viral fusion, infection, and/or replication of at least one highly pathogenic human coronavirus selected from SARS-CoV-1, MERS-CoV and SARS-CoV-2;

g) determining the ability of the selected antibodies, or antigen-binding fragments thereof, from f) to prevent or reduce infection in an in vivo model of HCoV infection selected from SARS-CoV-1, MERS-CoV and SARS-CoV-2; preferably determining the ability of the selected antibodies, or antigen-binding fragments thereof, to prevent or reduce infection in an in vivo model of HCoV infection from SARS-CoV-1, MERS-CoV, and SARS-CoV-2; and

h) selecting antibodies, or antigen-binding fragments thereof, that prevent or reduce infection in an in vivo model of HCoV infection selected from SARS-CoV-1, MERS-CoV and SARS-CoV-2; preferably selecting antibodies, or antigen-binding fragments thereof, that prevent or reduce infection in an in vivo model of HCoV infection from SARS-CoV-1, MERS-CoV and SARS-CoV-2.

In one aspect a method is provided for identifying a coronavirus cross-reacting antibody, said method comprising
providing PBMC samples from one or more human subjects, wherein the PBMC samples comprise B-cells selected from memory B-cells, plasma cells, or plasmablasts;
identifying B-cells that bind to at least part of the S (spike) protein from at least two, preferably at least four, different coronaviruses; preferably screening antibodies, or antigen-binding fragments thereof, for binding to at least part of the fusion peptide, the HR1 heptad repeat, or the HR2 heptad repeat of the S protein from at least two, preferably at least four, different coronaviruses
selecting antibodies, or antigen-binding fragments thereof, that bind to at least part of the S2 ectodomain of the S protein of at least one common human coronavirus selected from HCoV-NL63, HCoV-OC43, HCoV-229E and HCoV-HKU1, preferably HCoV-NL63 and that bind to at least part of the S2 ectodomain of the S protein of at least one highly pathogenic human coronavirus selected from SARS-CoV-1, MERS-CoV and SARS-CoV-2, preferably SARS-CoV-2; preferably wherein said selected antibody or antigen-binding fragment thereof also binds to at least part of the S2 ectodomain of the S protein of an animal coronavirus; preferably selecting antibodies, or antigen-binding fragments thereof, that bind to at least part of the S2 domain of the S protein of HCoV-NL63, HCoV-OC43, HCoV-229E and HCoV-HKU1; preferably selecting antibodies, or antigen-binding fragments thereof, that bind to at least part of S2 domain of the S protein of SARS-CoV-1, MERS-CoV and SARS-CoV2;
selecting antibodies or antigen-binding fragments thereof from above that inhibit viral fusion, infection, and/or replication of at least one common human coronavirus selected from HCoV-NL63, HCoV-OC43, HCoV-229E and HCoV-HKU1 and that inhibit viral fusion, infection, and/or replication of at least one highly pathogenic human coronavirus selected from SARS-CoV-1, MERS-CoV and SARS-CoV-2;
determining the ability of the selected antibodies, or antigen-binding fragments thereof to prevent or reduce infection in an in vivo model of HCoV infection selected from SARS-CoV-1, MERS-CoV and SARS-CoV-2; preferably determining the ability of the selected antibodies, or antigen-binding fragments thereof, to prevent or reduce infection in an in vivo model of HCoV infection from SARS-CoV-1, MERS-CoV, and SARS-CoV-2; and
selecting antibodies, or antigen-binding fragments thereof, that prevent or reduce infection in an in vivo model of HCoV infection selected from SARS-CoV-1, MERS-CoV and SARS-CoV-2; preferably selecting antibodies, or antigen-binding fragments thereof, that prevent or reduce infection in an in vivo model of HCoV infection from SARS-CoV-1, MERS-CoV and SARS-CoV-2.

Preferably, the method comprises providing a further plasma sample from a plurality of subjects, wherein said sample is collected at a time point (Y), wherein time point (Y) is at least 3 months earlier or later than time point (X).

Preferably, the plasma samples from a subject are selected which have an increase in immunoglobulins that bind to at least two HCoV's as compared to plasma samples from the subject collected at an earlier or later time point.

Preferably, the plasma samples have IgG, IgM, and/or IgA immunoglobulins that bind, independently, at least two HCoVs. Preferably, the immunoglobulins bind the S2 domain of a HCoV spike protein.

Preferably the method comprises:
selecting antigen-binding fragments that bind to at least part of the S2 domain of at least one common human coronavirus selected from HCoV-NL63, HCoV-OC43, HCoV-229E and HCoV-HKU1 and that bind to at least part of the S2 domain of the S protein of at least one highly pathogenic human coronavirus selected from SARS-CoV-1, MERS-CoV and SARS-CoV-2;

preferably wherein said selected antibody or antigen-binding fragment thereof also binds to at least part of the S2 ectodomain of the S protein of an animal coronavirus;
preparing IgM, IgA, or IgG antibodies comprising the selected antigen-binding fragments,
determining the ability of the IgM, IgA, or IgG antibodies to prevent or reduce infection in an in vivo model of HCoV infection selected from SARS-CoV-1, MERS-CoV and SARS-CoV-2; and
selecting IgM, IgA, or IgG antibodies that prevent or reduce infection in an in vivo model of HCoV infection selected from SARS-CoV-1, MERS-CoV and SARS-CoV-2.

In one aspect an antibody or antigen binding fragment thereof is provided which is identified or obtainable by the methods disclosed here. Preferably, the antibody is an IgG, IgM, or IgA antibody.

In one aspect a method of treating or preventing infection by a coronavirus, in particular infection by SARS-CoV-1, MERS-CoV or SARS-CoV-2, is provided comprising administering locally, preferably intranasally, to a subject in need thereof antibody or antigen binding fragment thereof as disclosed herein.

### BRIEF DESCRIPTION OF THE DRAWINGS

FIG. 1: Example of sorting strategy in FACS for NL63-S specific memory B-cells.

FIG. 2: ELISA results 293T supernatants. ELISA results of sort F NL63 S memory B-cell derived antibodies (expressed in small scale HEK293T cultures) for binding to NL63 S, SARS-COV-2 S, and SARS-COV-2 S2 trimer.

FIG. 3: FACS and ELISA results of NL63 S memory B-cell derived and purified monoclonal antibodies (expressed in larger scale HEK293F cultures). FIG. 3A). FACS results for sort B/E and sort F NL63-derived for binding to S protein of common hCoV NL63, hCoVs OC43 S and 229E (sort B/E only) and pathogenic hCoV SARS-CoV-2. FIG. 3B) ELISA results for binding to pathogenic hCoV SARS-CoV-2 S2 trimer.

### DETAILED DESCRIPTION OF THE DISCLOSED EMBODIMENTS

As a response to the SARS-CoV-2 pandemic, various efforts have been made to develop a SARS-CoV-2 specific vaccine in order to prevent infection. Typical vaccine development relies on using the disease-causing virus and either attenuating the virus for use as a ‘live attenuated vaccine’ or inactivating the virus. Subunit vaccines may also be developed based fragments of the virus, such as a surface protein. Vaccine development, particularly in relation to new viruses, is a long and difficult process. Vaccines may also be less effective in at-risk groups such as the elderly.

Efforts have also been made to identify antibodies directed to SARS-CoV-2 from COVID-19 patients. See, e.g. Kreer C et al. (Longitudinal isolation of potent near-germline SARS-CoV-2 neutralizing antibodies from COVID-19 patients. MedRxiv 2020) which describes the identification of neutralizing antibodies from COVID-19 patients.

In contrast, the solution provided by the approach described herein does not rely on SARS-CoV-2 neutralizing antibodies from COVID-19 patients. While not wishing to be bound by theory, it is proposed herein that while such antibodies isolated from COVID-19 patients may specifically target the SARS-CoV-2 strain that infected a particular patient, such antibodies are less effective at targeting evolving SARS-CoV-2 strains, other pathogenic coronaviruses, or animal coronaviruses susceptible to cross-species transmission. As demonstrated by the MERS-CoV, SARS-CoV-1, and SARS-CoV-2 outbreaks, cross-species transmission of coronavirus leads to disease emergence. Antibodies that are highly specific to a particular HCoV strain are unlikely to provide significant, if any, protection against such newly emergent HCoVs.

In some aspects, the disclosure provides method for identifying a coronavirus cross-reacting antibody. In one step, the methods comprise providing plasma samples from one or more (e.g., a plurality) human subjects. The samples are collected at a time point (X). The time point of collection is independent for each subject.

The human subjects have preferably never been infected with MERS-CoV, SARS-CoV-1, or SARS-CoV-2. Due to the relatively low number of infections from MERS-CoV and SARS-CoV-1, all samples collected prior to the end of 2019 in regions that were not affected with MERS-CoV and SARS-CoV-1 are likely to be from subjects that have never been infected with MERS-CoV, SARS-CoV-1, or SARS-CoV-2.

In some embodiments the subjects are at least 40 years, more preferably at least 45 years or older. Older individuals have a greater likelihood of having been infected by multiple HCoVs. In some embodiments the subjects are 75 years or younger, more preferably 65 years or younger. Preferably the subjects are between 45-65 years old.

As described in more detail below, the plasma sample is useful for, e.g., determining whether a subject is or has been infected by one or more HCoVs. In some embodiments, the methods further comprise selecting plasma samples collected from a subject that is or has been infected with a HCoV (preferably at least two common HCoV's). Accordingly, the methods preferably comprise selecting plasma samples having immunoglobulins that bind, human coronaviruses (HCoV), preferably at least two human coronaviruses, wherein the HCoV is selected from HCoV-NL63, HCoV-OC43, HCoV-229E and HCoV-HKU1. Otherwise stated, plasma samples may be selected having immunoreactivity to HCoV. Preferably, the plasma sample has immunoreactivity to at least three and at least four of the HCoVs selected from HCoV-NL63, HCoV-OC43, HCoV-229E and HCoV-HKU1. Preferably, the plasma sample has immunoreactivity to HCoV NL63. Preferably, the plasma sample has immunoreactivity to HCoV NL63 and HKU1.

Immunoreactivity and immunoglobulin binding refer to the ability of immunoglobulins in the plasma sample to recognize an HCoV as an antigen. It is not necessary in the present context that said immunoglobulins produce a measurable immune effect. Increased immunoglobulin binding to two viruses, e.g., HCoV-NL63 and HCoV-HKU1, may refer to the increase in immunoglobulins that bind to both HCoV-NL63 and HCoV-HKU1, but it is expected that such an increase will be due to an increase in particular immunoglobulins that bind to HCoV-NL63 and particular immunoglobulins that bind to HCoV-HKU1.

The sequences of “common” HCoVs are known in the art as are the sequences of the viral proteins encoded by said viruses. Sources of coronavirus may be a clinical isolate, e.g., obtained from a nasal or throat swab of a human patient. The virus may be propagated on a cell line, for example a mammalian cell line, such as vero cells, MadinDarby canine kidney (MDCK) cells, and PERC6 cells. Exemplary HCoV-NL63 sequences are described in WO2005017133. The genomic sequence of several clinical isolates are also publicly available; e.g., the genomic sequence of Human coronavirus NL63 isolate Amsterdam 496 is described in Pyrc et al. (J. Mol. Biol. 364 (5), 964-973 (2006) having accession number DQ445912 (VRL 21 Nov. 2006); the genomic sequence of Human coronavirus NL63 isolate Amsterdam 057 is described in Pyrc et al. (J. Mol. Biol. 364 (5), 964-973 (2006) having accession number DQ445911 (VRL 21 Nov. 2006); the genomic sequence of Human coronavirus NL63 isolate ChinaGD01 is described in Zhang et al. (Microbiol Resour Announc 9 (8), e01597-19 (2020)) having accession number MK334046 (28 Feb. 2020); the genomic sequence of Human coronavirus NL63 isolate ChinaGD05 is described in Zhang et al. (Microbiol Resour Announc 9 (8), e01597-19 (2020)) having accession number MK334045 (VRL 28 Feb. 2020); the genomic sequence of Human coronavirus NL63 isolate NL63/human/USA/891-4/1989 has accession number KF530114 (VRL 26 Sep. 2014); and the genomic sequence of Human coronavirus NL63 isolate NL63/human/USA/838-9/1983 has accession number KF530110 (VRL 26 Sep. 2014). A BLAST analysis of the six isolates listed above indicates that they share greater than 98% sequence identity.

The genomic sequence of several clinical isolates of HCoV-229E are publicly available; e.g., the genomic sequence of Human coronavirus 229E isolate 0349 is described in Farsani et al. (Virus Genes 45 (3), 433-439 (2012)) having accession number JX503060 (VRL 4 Apr. 2013); the genomic sequence of Human coronavirus 229E isolate J0304 is described in Farsani et al. (Virus Genes 45 (3), 433-439 (2012)) having accession number JX503061 (VRL 4 Apr. 2013); the genomic sequence of Human coronavirus 229E/Seattle/USA/SC9724/2018 has accession number MN369046 (VRL 21 Feb. 2020); the genomic sequence of Human coronavirus 229E/human/USA/933-40/1993 has accession number KF514433 (VRL 26 Sep. 2014); the genomic sequence of Human coronavirus 229E/BN1/GER/2015 has accession number KU291448 VRL (4 Sep. 2016); and the genomic sequence of Human coronavirus 229E/Seattle/USA/SC1212/2016 has accession number KY369911 (VRL 21 Feb. 2020). A BLAST analysis of the six isolates listed above indicates that they share greater than 99% sequence identity. In addition, the virus is also publicly accessible from ATCC as Human coronavirus 229E (ATCC VR-740; Hamre D, Procknow J J. A new virus isolated from the human respiratory tract. Proc. Soc. Exp. Biol. Med. 121: 190-193, 1966).

The genomic sequence of several clinical isolates of HCoV-HKU1 are publicly available; e.g., the genomic sequence of Human coronavirus HKU1 isolate Caen1 has accession number HM034837 (VRL 8 Oct. 2010); the genomic sequence of Human coronavirus HKU1 isolate genotype A has accession number AY597011 (VRL 27 Jan. 2006); the genomic sequence of Human coronavirus HKU1/human/USA/HKU1-15/2009 is described in Dominguez et al. (J. Gen. Virol. 95 (PT 4), 836-848 (2014)) having accession number KF686344 (VRL 26 Sep. 2014); the genomic sequence of Human coronavirus HKU1/human/USA/HKU1-5/2009 has accession number KF686340 (VRL 26 Sep. 2014); the genomic sequence of Human coronavirus HKU1/human/USA/HKU1-11/2009 has accession number KF430201 (VRL 26 Sep. 2014). A BLAST analysis of the six isolates listed above indicates that they share greater than 99% sequence identity.

The genomic sequence of several clinical isolates of HCoV-OC43 are publicly available; e.g., the genomic sequence of Human coronavirus OC43 isolate MDS16 has accession number MK303625 (VRL 30 Mar. 2019); the genomic sequence of Human coronavirus OC43 isolate MDS12 has accession number MK303623 (VRL 30 Mar. 2019); the genomic sequence of Human coronavirus OC43/Seattle/USA/SC9428/2018 has accession number MN310476 (VRL 21 Feb. 2020); the genomic sequence of Human coronavirus OC43/Seattle/USA/SC9430/2018 has accession number MN306053 (VRL 21 Feb. 2020); the genomic sequence of Human coronavirus OC43/human/USA/9211-43/1992 has accession number KF530097 (VRL 26 Sep. 2014); and the genomic sequence of Human coronavirus OC43/human/USA/873-6/1987 has accession number KF530087 (VRL 26 Sep. 2014). A BLAST analysis of the six isolates listed above indicates that they share greater than 98% sequence identity.

Methods for assaying immunoreactivity to HCoVs are known in the art. See, for example Chan K H et al. Serological Responses in Patients With Severe Acute Respiratory Syndrome Coronavirus Infection and Cross-Reactivity With Human Coronaviruses 229E, OC43, and NL63. Clin Diagn Lab Immunol. 2005 November; 12(11):1317-21; Kramer A R et al. The Human Antibody Repertoire Specific for Rabies Virus Glycoprotein as Selected From Immune Libraries. Eur J Immunol. 2005 Jul; 35(7):2131-45; and Pohl-Koppe 1995 Journal of Virological Methods 55:175-183. Such methods include, e.g., using viral proteins, or fragments thereof, in order to detect immunoglobulins present in plasma samples using, e.g., Western blot analysis, ELISA's, or any other known immunoassays. Preferably, the methods comprise determining (qualitatively or quantitatively) the presence of immunoglobulins that bind HCoV. Preferably, the methods comprise determining the immunoreactivity of the plasma samples to one or more HCoVs.

As will be apparent to a skilled person, immunoreactivity or antibody binding to HCoV includes immunoreactivity or antibody binding to a protein encoded by said virus. Preferably, the methods select plasma samples having an increase in immunoglobulins that bind the S2 domain of a HCoV S protein.

While not wishing to be bound by theory, immunoreactivity to HCoV is believed to be an indicator of infection or past infection by the HCoV. Preferably, the methods detect IgM, IgA, or IgG. More preferably IgM and IgA are detected as these immunoglobulins are indicative of early infection. A skilled person is readily able to determine whether the level of immunoglobulins that bind HCoV indicates infection or if it is within background levels. For example, immunoreactivity is indicated if antibodies can be detected in plasma that is at least 8-fold diluted.

In some embodiments of the methods, further plasma samples may be collected from the same subject over time. For example, samples may be collected every 3, 6, 9, or 12 months or any combination thereof. In some embodiments, the methods comprise providing a plasma sample collected at a time point (Y), wherein time point (Y) is at least 3 months earlier or later than time point (X).

Samples collected at various time points from the same subject provides the advantage that viral infection of a subject at a particular time point can be determined by comparing HCoV specific immunoglobulin levels between samples. For example, an increase of HCoV-NL63 specific immunoglobulin levels in plasma as compared to samples collected at previous time points would indicate that the subject was infected (or recently infected) with HCoV-NL63 at the time said sample was collected.

A skilled person can readily determine whether the difference in immunoglobulin levels is “significant”. For example, while a 10% increase in IgM levels may be considered significant, smaller increases (e.g., 5%) in both IgM and IgA levels may be considered significant.

Subjects having plasma samples at a time point (X) with immunoreactivity to at least two HCoVs are likely to have been infected with at least two HCoVs at or before time point (X). While not wishing to be bound by theory, the disclosure provides that B-cells from some of these subjects may encode cross-reactive pan-corona antibodies. Accordingly, the method further discloses providing PBMC samples from said subjects. The PBMC samples are collected at time point (X) or later. A skilled person will recognize that “at time point (X)” also includes a few days prior to time point X.

As will be understood by the skilled person, a plasma sample refers to a sample comprising plasma and a PBMC sample refers to a sample comprising PBMCs. While the plasma sample and the PBMC sample may be the same sample such as a blood sample (e.g., collected at time point (X)), for long-term storage these two components are normally separated. In one embodiment, a blood sample may be obtained from a subject at a time point X. The blood sample may be further processed in order to prepare a blood plasma sample and a PBMC sample which can be stored separately for long periods of time, if needed. Methods for processing plasma and PBMCs for storage are well-known in the art.

In some embodiments, the methods comprise providing a plasma sample from a subject and determining immunoreactivity as described herein. PBMC samples may then be obtained from subjects whose plasma samples showed immunoreactivity to at least two HCoVs. In some embodiments, the methods comprise providing samples from a plurality of human subjects, wherein for each subject the samples comprise a pair of samples comprising a plasma sample and a peripheral blood mononuclear cell (PBMC) sample collected at a time point (X). In some embodiments, the methods comprise providing at least a second pair of samples from a subject comprising a plasma sample and a peripheral blood mononuclear cell (PBMC) sample collected at a time point (Y), wherein time point (Y) is at least 3 months earlier or later than time point (X).

The method comprises providing PBMC samples containing memory B cells. As used herein, “memory B-cells” refers to CD27+/IgA+; CD27+/IgG+; CD27+/IgM+ and CD27+/IgM+/IgD+ memory B cells that may furthermore be CD19+, CD22+ and/or CD24+.

In some embodiments, the memory B-cells may be isolated or enriched from the other PBMCs. The characteristics of memory B-cells as well as methods for isolating or enriching for such cells are known in the art. For example, memory B-cells can be enriched by positive cell sorting using anti-CD27 microbeads.

The method further comprises screening the antibodies encoded by the memory B-cells for binding to at least a part of the S2 ectodomain from different coronaviruses. While in some cases antibodies from all memory B-cells may be screened, the disclosure encompasses using only fractions of the B-cells. In some embodiments, the entire S protein or the S2 ectodomain from several different coronaviruses are used to screen the antibodies. In some embodiments, peptides corresponding to a domain selected from the UH, FP, HR1, central helix, beta-hairpin, and HR2 domains are used to screen for antibody binding. In some embodiments, peptides correspond to a domain selected from the FP, HR1 or HR2 domain. The peptides need not correspond to an entire domain as described above, but also include peptides that contain at least a part of the domains. In some embodiments, the peptides are between 10-200 amino acids, preferably between 12 to 50 amino acids. In some embodiments, the step of screening antibodies, or antigen-binding fragments thereof, binding to at least part of the S2 ectodomain refers to performing binding assays to identify antibodies that bind to at least part of the S2 ectodomain.

In some embodiments, antibodies are screened, and preferably selected, for binding to at least one common human coronavirus selected from HCoV-NL63, HCoV-OC43, HCoV-229E and HCoV-HKU1 and at least one highly pathogenic human coronavirus selected from SARS-CoV-1, MERS-CoV and SARS-CoV-2. In some embodiments, antibodies are screened, and preferably selected, for binding to HCoV-NL63, HCoV-OC43, HCoV-229E and HCoV-HKU1. In some embodiments, antibodies are screened, and preferably selected, for binding to SARS-CoV-1, MERS-CoV and SARS-CoV-2. In some embodiments, antibodies are screened, and preferably selected, for binding to non-human coronavirus, e.g., a swine, cattle, horses, camels, cats, dogs, rodents, birds, bats, rabbits, ferrets, or mink coronavirus. Preferably, the antibodies are screened for binding 5 or more or even 10 or more different coronaviruses.

In a preferred embodiment, the antibodies are screened for binding to the S2 ectodomain from at least one highly pathogenic HCoV, at least one common HCoV, and at least one animal CoV. In some embodiments, antibodies are selected which bind to at least one common human coronavirus selected from HCoV-NL63, HCoV-OC43, HCoV-229E and HCoV-HKU1; at least one highly pathogenic human coronavirus selected from SARS-CoV-1, MERS-CoV and SARS-CoV-2; and at least one animal coronavirus selected from swine, cattle, horses, camels, cats, dogs, rodents, birds, bats, rabbits, ferrets, or mink coronavirus. See, e.g., Fenner's Veterinary Virology, Chapter 24-Coronaviridae 2017, pp. 435-461 and for sequences the CoVDB (Coronavirus Database at covdb.popgenetics.net/v1/).

In an exemplary embodiment of the disclosure, antibodies are screened to determine binding to the S2 ectodomain of HCoV-NL63, HCoV-OC43, HCoV-229E, HCoV-HKU1, SARS-CoV-1, MERS-CoV, SARS-CoV-2, and at least one animal coronavirus. Preferred antibodies demonstrate binding to all of the tested S2 ectodomains. However, a skilled person will appreciate that antibodies binding to only a subset are also useful as cross-reactive antibodies.

As will be clear to a skilled person, the respective antigen-binding fragments may also be used in the screening step. Antigen-binding fragments of antibodies include Fab, F(ab′)2, and Fv fragments. Preferably, an antigen-binding fragment comprises CDRs 1-3 of the heavy chain and CDRs 1-3 of the light chain More preferably, an antigen-binding fragment comprises the light and heavy chain variable regions.

For example, in some embodiments a single chain variable fragment (ScFv) phage display antibody library may be constructed using the antibody sequences (i.e., variable chain domains) encoded by the memory B-cells. See, for example, Kramer A R et al. The Human Antibody Repertoire Specific for Rabies Virus Glycoprotein as Selected From Immune Libraries. Eur J Immunol. 2005 July; 35(7):2131-45). Phage display libraries are then screened for phage binding to an antigen (corresponding to the S2 domain of the S protein) through (bio-)panning.

In some embodiments, single cell screening may be performed in order to identify B-cells that bind to a particular target. This can be used as an initial step prior to, e.g., generating a phage display library. In some embodiments, B-cells are screened for their ability to bind the S2 ectodomain from a panel of coronaviruses. Positive B-cells are then used as a source for generating a phage display library in order to identify antigen binding fragments that cross-react with multiple coronaviruses.

In other embodiments, the antibodies produced by said B-cells may be screened. For example, in some embodiments a B-cell is immortalized and the secreted antibody is screened against said panel. B-cells can be immortalized by, e.g., infecting B-cells with the Epstein Barr Virus (EBV) and individual clones can be grown. Efficiency of immortalization and cloning of EBV-immortalized cells can be improved by also using an agonist of a Pattern Recognition Receptor that is expressed on memory B cells, e.g., TLR-7, TLR-9 or TLR-10 agonists (see, e.g., U.S. Pat. No. 9,290,786B2) Methods for screening immunoglobulins to determine antigen binding are known in the art. Western blot analysis, ELISA's, or any other known immunoassay may be used. For example, peptides may be linked to solid surfaces such as peptide microarrays (i.e., peptide chips). In some embodiments, Pepscan analysis can be performed, for example, where overlapping 15-mer linear peptides spanning the S2 domain are screened for immunoglobulin binding (see, e.g., Kramer et al. The Human Antibody Repertoire Specific for Rabies Virus Glycoprotein as Selected From Immune Libraries. Eur J Immunol. 2005 July; 35(7):2131-45). The S2 domain or parts thereof may also be expressed on cell surfaces and used to screen for immunoglobulin binding.

B-cells which are identified as encoding or expressing an antibody that binds the S2 domain may be used as the source of nucleic acid for cloning the antibody genes.

The method further comprises selecting antibodies, or antigen-binding fragments thereof, that bind to at least a part of the S2 ectodomain of the S protein of at least one common human coronavirus selected from HCoV-NL63, HCoV-OC43, HCoV-229E and HCoV-HKU1 and that bind to the S2 domain of the S protein of at least one highly pathogenic human coronavirus selected from SARS-CoV-1, MERS-CoV and SARS-CoV-2. In a preferred embodiment, antibodies, or antigen-binding fragments thereof, are selected which bind to the HR1 domain of HCoV-NL63, the HR1 domain of HCoV-OC43, the HR1 domain of HCoV-229E and the HR1 domain of HCoV-HKU1 and/or bind to the HR1 domain of SARS-CoV-1, the HR1 domain of MERS-CoV and the HR1 domain of SARS-CoV-2.

The method further comprises selecting antibodies or antigen-binding fragments thereof that inhibit viral fusion, replication, and/or replication of at least one common human coronavirus selected from HCoV-NL63, HCoV-OC43, HCoV-229E and HCoV-HKU1 that inhibit viral fusion and/or inhibit cell infectivity of at least one highly pathogenic human coronavirus selected from SARS-CoV-1, MERS-CoV and SARS-CoV-2. Viral fusion assays, infection assays, and replication assays are well-known to the skilled person and exemplary methods for performing such methods are described herein in the examples. For example, multiple cell-cell fusion assays that are mediated by the S protein of various HCoVs have been developed (Xia S, Yan L, Xu W, et al. A pan-coronavirus fusion inhibitor targeting the HR1 domain of human coronavirus spike. Sci Adv. 2019; 5(4)). Pseudotyped virus infection assays, such as those described in Lu et al. (Nat. Commun 5, 3067 (2014), may also be used. Assays to measure HCoV replication have also been described (see examples). As will be clear to a skilled person, complete inhibition is not required and a skilled person is able to identify antibodies that significantly inhibit viral fusion, infection, or replication. Preferably, antibodies result in at least 50% inhibition.

In some embodiments, the methods comprise measuring the effect of the antibodies or antigen-binding fragments on viral fusion, infection, and/or replication. In an exemplary embodiment of the disclosure, antibodies are screened to determine the effect on fusion, infection, or replication with HCoV-NL63, HCoV-OC43, HCoV-229E, HCoV-HKU1, SARS-CoV-1, MERS-CoV, SARS-CoV-2, and at least one animal coronavirus. Preferred antibodies inhibit fusion, infection, and/or replication of HCoV-NL63, HCoV-OC43, HCoV-229E, HCoV-HKU1, SARS-CoV-1, MERS-CoV, SARS-CoV-2, and at least one animal coronavirus. However, a skilled person will appreciate that antibodies inhibiting fusion, infection, and/or replication to only a subset of coronaviruses are also useful as cross-reactive antibodies.

The method further comprises determining the ability of the selected antibodies, or antigen-binding fragments thereof, to prevent or reduce infection in an in vivo model of HCoV infection selected from SARS-CoV-1, MERS-CoV and SARS-CoV-2. Antigen-binding fragments, such as scFv's may be used in the in vivo models. Preferably, full-length antibodies in the IgG, IgM, or IgA format are used. Methods for cloning variable regions into full-length formats or into different full-length formats are known in the art. See, e.g., Boel E et al. Functional Human Monoclonal Antibodies of All Isotypes Constructed From Phage Display Library-Derived Single-Chain Fv Antibody Fragments. J Immunol Methods. 2000 May 26; 239(1-2):153-66. In some embodiments, the antigen-binding fragment from a particular antibody may be tested in several formats, such as in the IgG, IgM, and IgA format in order to determine whether the antibody format has an effect on function.

In vivo models of SARS-CoV-1, MERS-CoV and SARS-CoV-2 infection are known. Suitable in vivo models of SARS-CoV-2 infection are described in, e.g., Sia, S. F., Yan, L., Chin, A. W. H. et al. Pathogenesis and transmission of SARS-CoV-2 in golden hamsters. Nature (2020). Suitable in vivo models of MERS-CoV infection are described in, e.g., Kim J et al. Middle East Respiratory Syndrome-Coronavirus Infection Into Established hDPP4-Transgenic Mice Accelerates Lung Damage Via Activation of the Pro-Inflammatory Response and Pulmonary Fibrosis. J Microbiol Biotechnol. 2020 Mar. 28; 30(3):427-438. Suitable in vivo models of SARS-CoV-1 infection are described in, e.g., Roberts et al. Virus Research 2008 133:20-32.

The prevention or reduction of infection in an in vivo includes increased resistance to infection or an improved ability to fight infection (e.g., infection may be cleared before symptoms arise or symptoms experienced are milder). Mortality, weight loss, and lung pathology may be used as indicators of the ability to prevent or reduce infection in vivo.

In one aspect, the disclosure provides a coronavirus cross-reacting antibody. The antibody binds to at least one, preferably at least two, HCoVs selected from SARS-CoV-1, MERS-CoV and SARS-CoV-2. The antibody also binds to at least one, at least two, at least three, or at least four HCoVs selected from HCoV-NL63, HCoV-OC43, HCoV-229E and HCoV-HKU1. Preferably, the antibody also binds at least one or more animal coronaviruses such as a swine, cattle, horses, camels, cats, dogs, rodents, birds, bats, rabbits, ferrets, or mink coronavirus. Preferably, the antibody binds to the S2 domain of the S protein. Preferably, the antibody binds to the fusion peptide, HR1, or HR2 of the S protein. In some embodiments, the antibody is identified according to a method as disclosed herein.

The antibodies identified herein are useful for increasing immunity in an individual against a coronavirus. The term “increasing immunity” refers to increasing an individual's immune response against a particular antigen (e.g., coronavirus). Increased immunity can lead to increased resistance to infection or may improve an individual's ability to fight infection (e.g., infection may be cleared before symptoms arise or symptoms experienced are milder). Increased immunity does not require full immunity, but also includes partial immunity. As will be clear to a skilled person, the methods and compositions disclosed herein may be used to prevent or reduce coronaviral infection and/or reduce the severity of coronaviral infection. Said methods and compositions may also be used to prevent or reduce the severity of symptoms associated with coronaviral infection. In particular, increased immunity refers to passive immunity, such as by the administration of antibodies.

In some embodiments, increased immunity refers to providing sterilizing immunity. In contrast to immunity that allows for infection but is effective at clearing the infection, sterilizing immunity prevents an effective viral infection. In some embodiments, the compositions disclosed herein provide sterilizing immunity. In some embodiments, compositions comprising an antibody as disclosed herein provide sterilizing immunity for at least one day, preferably at least one week. In some embodiments, compositions comprising an antibody as disclosed herein provide sterilizing immunity for 1-2 weeks.

The disclosure provides methods and compositions for increasing immunity against a coronavirus. Preferably, the individual is a human and the coronavirus is a human coronavirus, i.e., a virus capable of infecting humans. Preferably, the coronavirus is a highly pathogenic virus, or rather a virus which can lead to severe symptoms in infected patients. In some embodiments, a highly pathogenic virus as used herein refers to a virus having a fatality rate of 1% are higher. Exemplary highly pathogenic coronaviruses include MERS-CoV (fatality rate of around 34%), SARS-CoV-1 (fatality rate of around 9.5%), and SARS-CoV-2 (fatality rate of around 2%) (Petrosillo et al. Clinical Microbiology and Infection Volume 26, Issue 6, June 2020, Pages 729-734). Virulence can also be defined based on the severity of symptoms. For example, in some embodiments, a highly pathogenic coronavirus as used herein refers to a virus that causes acute respiratory distress syndrome in at least 10% of infected individuals, which includes SARS-CoV-1, SARS-CoV-2, and MERS-CoV (Petrosillo et al. 2020). In preferred embodiments, the coronavirus is an alpha- or beta-coronavirus.

In some embodiments, the disclosure provides nucleic acid molecule encoding antibodies disclosed herein. A further aspect of the disclosure provides vectors and expression vectors comprising the nucleic acid molecules disclosed herein. Expression vectors useful in the present disclosure include vaccinia virus, retroviruses, and baculovirus. The expression vector may comprise the nucleic acid sequences disclosed herein or a fragment thereof that is under control of or operatively linked to a regulatory element, such as a promoter. The segment of DNA referred to as the promoter is responsible for the regulation of the transcription of DNA into mRNA. The expression vector may comprise one or more promoters suitable for the expression of the gene in, e.g., plant cells, fungal cells, bacterial cells, yeast cells, insect cells or other eukaryotic cells.

Compositions comprising the antibodies described herein can be formulated together with a pharmaceutically acceptable carrier, diluent and/or adjuvant. Examples of pharmaceutically acceptable carriers or diluents include demineralised or distilled water; saline solution; vegetable-based oils, cellulose derivatives, polyethylene glycol, etc.

Preferably, the antibodies disclosed herein are administered locally, or rather not systemically. Local administration includes administration to the skin, eyes, and mucosal membranes. In preferred embodiments, the compositions are applied to mucous membranes such as the bronchial, esophageal, nasal, and oral mucosa and the tongue. Preferably, the composition is provided by nasal inhalation. The composition may also be provided to the nose as a crème or lotion. The composition may also be inhaled by mouth or applied to the mucosal membranes of the mouth, for example as a mouthwash. In some embodiments, the compositions are not administered parenterally (e.g., by not by intravenous (IV), intramuscular (IM), subcutaneous (SC) or intradermal (ID) administration).

The compositions may be administered prophylactically or “as needed” before potential encounters with a highly pathogenic coronavirus. For example, the compositions may be administered once daily Administration of the composition may be skipped on days where the individual remains at home with no risk of exposure.

As used herein, “to comprise” and its conjugations is used in its non-limiting sense to mean that items following the word are included, but items not specifically mentioned are not excluded. In addition, the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 1% of the value.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms. Treatment may also be continued after symptoms have resolved, for example to prevent or delay their recurrence. As used herein, the term “prevent” does not require the absolute prevention of, e.g., infection but may reduce the risk or likelihood of infection.

All patent and literature references cited in the present specification are hereby incorporated by reference in their entirety.

### EXAMPLES

The invention is further explained in the following examples. These examples do not limit the scope of the invention, but merely serve to clarify one possible way of carrying out the invention. A skilled person will recognize that other methods may be used.

### Example 1

Plasma and PBMC samples are obtained from several human subjects as follows.

From each donor, 10 ml of blood is collected in Vacutainer tubes (10 ml) supplemented with sodium heparin as anti-coagulant. Within eight hours after collection plasma and peripheral blood mononuclear cells (PBMC) are isolated by using Ficoll-Paque (GE Healthcare Bio Sciences AB, Uppsala, Sweden) density-gradient centrifugation in accordance with the manufacturer's instructions. Plasma is harvested and transferred into one milliliter Nalgene cryotubes (Nalgene)

The buffy coat, containing the PBMNC is harvested and washed three to five times in cold (+4° C.) phosphate buffered saline (PBS) to get rid of the thrombocytes present in the buffy coat. After the last wash step, the cell pellet containing the PBMNC is diluted with cold freezing medium containing 10% dimethyl sulfoxide (DMSO), 40% heat inactivated fetal calf serum (FCS), and 50% RPMI 1640 culture medium at a concentration of 5×10⁶ cells/ml. Freezing medium was added to cell suspension dropwise under continuous mixing. The cell suspension is transferred into one milliliter Nalgene cryotubes

Cryotubes containing plasma or PBMNC cell suspension, are placed into a Mr. Frosty freezing container (Nalgene) and kept overnight at −80° C. before being transferred into liquid nitrogen for long-term storage.

### Example 2

Plasma samples are tested for immunoreactivity to HCoVs using one or more of the following methods.

1. ELISA Test

Antibody levels to the S, N and/or c-terminal of N (NCt) proteins of HCoV-OC43, HCoV-229, HCoV-NL63 and HCoV-HKU1 are determined by ELISA. Hereto, the S, N and NCt sequences of HCoV-OC43, HCoV-229, HCoV-NL63 and HCoV-HKU1 are cloned, expressed and purified as described earlier (Dijkman R, Jebbink M F, El Idrissi N B, Pyrc K, Muller M A, Kuijpers T W, et al. Human coronavirus NL63 and 229 E seroconversion in children. J Clin Microbiol 2008; 46(July (7)):2368-73; Dijkman R, Jebbink M F, Gaunt E, et al. The dominance of human coronavirus OC43 and NL63 infections in infants. J Clin Virol. 2012; 53(2):135-139). ELISAs are performed as described earlier (Edridge et al., Coronavirus protective immunity is short-lasting, medRxiv 2020.05.11.20086439). Briefly, ninety-six half area microplates (Greiner Bio-one) are coated overnight at 4° C. with 3 μg/mL protein diluted in 0.1 M carbonate buffer pH 9.6. Non-specific binding sites are blocked with phosphate-buffered saline-0.1% Tween 20 (PBST) supplemented with 5% skim milk (Fluka), mildly shaking for 1 hour at room temperature. Serum samples are diluted 1:200 in PBST containing 1% skim milk and incubated in the plate, mildly shaking for 2 hours at room temperature. After a washing, alkaline phosphatase-conjugated anti-human immunoglobulin G Fcγ-tail antibody (Jackson Immunoresearch) diluted (1:1500) in 1% skim milk-PBST is added. Following a mildly shaking 1 hour incubation at room temperature, the plates are washed and signal developed with Lumi-Phos Plus (Lumigen), 1 hour mildly shaking in the dark at room temperature. Measurements are done with a Glomax 96 plate luminometer (Promega). All sera are tested in duplicate or triplicate and normalized to correct for differences in lumination times. For each normalized observation, standard deviations are calculated between technical replicates (duplicate/triplicate ELISA's are performed on newly made dilutions of the same serum sample).

2. Pepscan Analysis.

15-mer peptides, overlapping by 14 residues, from the S protein sequences of HCoV-OC43, HCoV-229, HCoV-NL63 and HCoV-HKU1 are synthesized by Fmoc coupling on the solid support of a Pepscan hydrogel (Langedijk J P M, Zekveld M J, Ruiter M, Corti D, Back J W. Helical peptide arrays for lead identification and interaction site mapping. Anal. Biochem. 2011; 417:149-155. [PubMed: 21708118]). The peptide libraries are probed with heat-inactivated human sera, at a 1:1000 dilution. After extensive washing, a goat anti-human HRP conjugated secondary antibody is added, followed by color development using 2,2′-azino-bis(3-ethylbenzothiazoline-6-sulphonic acid). A charge-coupled device camera is used to quantify the absorbance at 405 nm. For every individual Pepscan dataset, the data is normalized to the average signal intensity derived from the overall analysis.

3. Testing AB Titer Using HCoV

Antibodies titers against HCoV-OC43, HCoV-229, HCoV-NL63 and HCoV-HKU1 were determined by immunofluorescent test as described earlier, with some modifications (Chan K H et al. Serological Responses in Patients With Severe Acute Respiratory Syndrome Coronavirus Infection and Cross-Reactivity With Human Coronaviruses 229E, OC43, and NL63. Clin Diagn Lab Immunol. 2005 November; 12(11):1317-21.)

Briefly HCoV-OC43 and HCoV-HKU1-infected HCT-8, HCoV-229-infected MRC-5 cells, and HCoV-NL63-infected LLCMK2 cell smears are used for the study. When 60% to 70% of cells have evidence of viral antigen expression, the cells are fixed in chilled acetone for 10 min at 20° C. and are stored at 80° C. until use. Antibody detection is performed using indirect immunofluorescence as described in article above. Plasma samples are selected which demonstrate immunoreactivity to least two HCoVs.

Donors are selected from which plasma samples demonstrate immunoreactivity to the S protein, S2 domain, or N protein domain from least two HCoVs.

### Example 3

PMBC samples from donors from which plasma samples demonstrate immunoreactivity to the S or N proteins from least two HCoVs as determined in Example 2 are used as a source of memory B-cells for isolation of antibodies using standard phage-display, B-cell immortalization or single-B-cell sorting approaches, such as those described below.

Selection of Memory B Cells

B-cells are enriched from PBMC samples. Memory B-Cells are labeled with specific fluorescence conjugated antibodies and sorted with a flow cytometer as descried earlier (Throsby M et al. Heterosubtypic Neutralizing Monoclonal Antibodies Cross-Protective Against H5N1 and H1N1 Recovered From Human IgM+ Memory B Cells. PLoS One. 2008; 3(12):e3942, Ellebedy A H et al. Defining antigen-specific plasmablast and memory B cell subsets in blood following viral infection and vaccination of humans. Nat Immunol. 2016; 17(10):1226-1234, Wrammert J et al. Rapid and Massive Virus-Specific Plasmablast Responses During Acute Dengue Virus Infection in Humans J Virol. 2012 March; 86(6):2911-8, Pascual G et al. Immunological Memory to Hyperphosphorylated Tau in Asymptomatic Individuals. Acta Neuropathol 2017 May; 133(5):767-783.) Sorted cells are collected as cell fraction or as single cells).

### Example 3A

In some experiments, the B-cells are used for the construction of a phage display antibody library as follows. Single chain variable fragment (ScFv) phage display libraries are constructed as described previously (Kramer A R et al. The Human Antibody Repertoire Specific for Rabies Virus Glycoprotein as Selected From Immune Libraries. Eur J Immunol. 2005 July; 35(7):2131-45). Briefly, phage libraries are constructed using antibody genes isolated from memory B-cells. The quality of the library is verified by analyzing randomly picked clones by using colony PCR.

Phage display selection of S2 domain-specific scFv phages is performed essentially as described earlier (Kramer A R et al. The Human Antibody Repertoire Specific for Rabies Virus Glycoprotein as Selected From Immune Libraries. Eur J Immunol. 2005 July; 35(7):2131-45.) but using panels of overlapping peptides spanning the S2 domains (in particular the UH, FP, HR1, central helix, beta-hairpin, and HR2 regions) of HCoV-OC43, HCoV-229, HCoV-NL63, HCoV-HKU1 as well as from SARS-CoV, MERS-CoV and SARS-CoV-2. Alternatively, cell-surface expressed or purified Spike proteins from HCoV-OC43, HCoV-229, HCoV-NL63, HCoV-HKU1 as well as from SARS-CoV, MERS-CoV and SARS-CoV-2 may be used.

Clones that demonstrate binding to peptide(s) (or surface-expressed or purified S protein) from at least one common human coronavirus selected from HCoV-NL63, HCoV-OC43, HCoV-229E and HCoV-HKU1 and at least one highly pathogenic human coronavirus selected from SARS-CoV-1, MERS-CoV and SARS-CoV-2 are selected.

### Example 3B

In some experiments, the B-cells are used for B-cell immortalisation for the production of monoclonal antibodies. Monoclonal antibody producing immortalised B-cell clones are generated as described previously (Lanzavecchia A et al. Human monoclonal antibodies by immortalization of memory B cells. Curr Opin Biotechnol. 2007; 18(6):523-528.)

When using memory B-cells, the cells must first be induced to generate plasma cells. Plasma cells are generated in vitro from memory B-cells as described earlier (Maïga R I et al Human CD38hiCD138⁺ Plasma Cells Can Be Generated in Vitro From CD40-activated Switched-Memory B Lymphocytes. J Immunol Res. 2014; 2014:635108). Briefly, sorted memory B-cells are expanded and subsequently cultured on CD154⁺ and CD70⁺ Adherent Cells, which generates CD38^{hi}CD138⁺ plasma cells.

Culture supernatants are tested for binding to peptides spanning the S2 domains and/or surface-expressed or purified S proteins from multiple coronaviruses as described above. B-cells that produce antibodies that bind to peptide(s) or S protein from at least one common human coronavirus selected from HCoV-NL63, HCoV-OC43, HCoV-229E and HCoV-HKU1 and at least one highly pathogenic human coronavirus selected from SARS-CoV-1, MERS-CoV and SARS-CoV-2 are selected.

### Example 3C

In some experiments, memory B-cells are selected that bind to the S2 domain in vitro. Memory B-cells that bind to viral peptides are selected essentially as described previously (Pascual G et al. Acta Neuropathol. 2017; 133(5): 767-783 Immunological memory to hyperphosphorylated tau in asymptomatic individuals)

Briefly, HR1, HR2 and/or FP domain peptide sequences from the spike protein of HCoV-NL63, HCoV-OC43, HCoV-229E, HCoV-HKU1, SARS-CoV, MERS-CoV and SARS-CoV-2 are prepared and biotinylated and conjugated to a fluorescent dye. CD22+ PBMNC cells are labeled with fluorescent conjugated antibodies IgG-FITC, CD19-PerCPCy5.5, and incubated with the biotinylated fluorescent viral peptides. Cells with memory B-cell phenotype that bind the fluorescent viral peptide are sorted as single cells with a flow cytometer.

Selected are used to generate full-length antibodies as described in Example 4. Such antibodies can then be further tested for binding to the S2 ectodomain from several coronaviruses.

Cells that bind peptides from at least one common human coronavirus selected from HCoV-NL63, HCoV-OC43, HCoV-229E and HCoV-HKU1 and at least one highly pathogenic human coronavirus selected from SARS-CoV-1, MERS-CoV and SARS-CoV-2 are selected.

### Example 4

IgG, IgA and/or IgM antibodies from selected phages (Example 3A) or memory B cells (Examples 3B and C) are generated.

Production of human IgG, IgM or IgA monoclonal antibodies from selected phages is performed as described earlier (Boel E et al. Functional Human Monoclonal Antibodies of All Isotypes Constructed From Phage Display Library-Derived Single-Chain Fv Antibody Fragments. J Immunol Methods. 2000 May 26; 239(1-2):153-66.)

Briefly, vectors are constructed for the production of human IgG1-4, IgA1-2, and/or IgM monoclonal antibodies. The V_{H} 3 H chain and V_{λ}3 L chain genes encoding scFv fragments of selected phages are cloned into the different expression vectors to generate monoclonal antibodies of the different Ig subclasses. Stably transfected cell lines are established by co-transfection of H and L chain constructs in fur-BHK21 cells. Culture supernatant is harvested and all subclasses are purified by using a protein A column.

IgG1-4, IgA1-2, IgM and IgE antibodies are essentially generated from selected memory B cells as described earlier (Apetri A et al. A Common Antigenic Motif Recognized by Naturally Occurring Human V H 5-51/V L 4-1 Anti-Tau Antibodies With Distinct Functionalities. Acta Neuropathol Commun 2018 May 31; 6(1):43).

Briefly, heavy and light chain (HC/LC) antibody variable regions are recovered using a two-step PCR approach from single cell sorted memory B cells using a pool of leader specific and framework specific primers Heavy and light chain PCR fragments (380-400 kb) are linked via an overlap extension PCR and subsequently cloned into a dual-CMV-based human IgG1-4, IgA1-2, IgM or IgE mammalian expression vector. Cloned anti-spike protein human monoclonal Antibodies are transiently transfected in human embryonic kidney 293-derived Expi293 cells (Thermo Fisher) and 72 h post transfection, cell media are harvested and centrifuged for 7 min at 1200 RPM Immunoglobulins are purified from the culture medium by standard Protein A affinity chromatography methods.

The monoclonal antibodies may be tested for binding to the S protein, S2 ectodomain or specific peptides from several coronaviruses such as HCoV-NL63, HCoV-OC43, HCoV-229E, HCoV-HKU1, SARS-CoV, MERS-CoV and SARS-CoV-2.

### Example 5

In vitro functionality of the monoclonal antibodies may be assessed using one or more of the following methods.

1. Cell-Fusion Inhibition.

Multiple cell-cell fusion assays that are mediated by the S protein of various HCoVs have been developed (Xia S, Yan L, Xu W, et al. A pan-coronavirus fusion inhibitor targeting the HR1 domain of human coronavirus spike. Sci Adv. 2019; 5(4). Specifically: (1) MERS-CoV S-mediated cell-fusion; (2) 229E S-mediated cell-fusion; (3) SARS-CoV and SL-CoV S-mediated cell-cell fusion; and (4) OC43 or NL63 S-mediated cell-cell fusion. To assess the inhibitory potency of the monoclonal antibodies against fusion mediated by the different S proteins, effector cells (293 T/S/GFP) and target cells (Huh-7 cells) are co-cultured in the presence or absence of a test mAb at the indicated concentrations for fusion. After counting the fused and unfused cells, the percentage of cell-cell fusion is calculated. Hereto, five fields in each well are randomly selected for counting the fused and unfused cells. The fused cells are at least twice as large as the unfused cells, and the fluorescence intensity in the fused cell becomes weak as a result of the diffusion of enhanced green fluorescent protein (EGFP) from one effector cell to target cells. The percentage of cell-cell fusion [(number of the fused cells/number of the fused and unfused cells)×100%] is then calculated. The percent inhibition of cell-cell fusion is calculated using the following formula: [1−(E−N)/(P−N)]×100%. Where “E” represents the percentage of cell-cell fusion in the experimental group. “P” represents the percentage of cell-cell fusion in the positive control group, in which 293 T/HCoV S/EGFP cells are used as effector cells, to which only PBS was added. “N” is the percentage of cell-cell fusion in negative control group, in which 293 T/EGFP cells are used as effector cells.

2. Pseudotyped Virus Infection Assay

A pseudovirus bearing CoV S protein or VSV-G protein and a defective HIV-1 genome that expresses luciferase as reporter is produced in 293 T cells, as previously described ((L. Lu, Q. Liu, Y. Zhu, K.-H. Chan, L. Qin, Y. Li, Q. Wang, J. F.-W. Chan, L. Du, F. Yu, C. Ma, S. Ye, K.-Y. Yuen, R. Zhang, S. Jiang, Structure-based discovery of Middle East respiratory syndrome coronavirus fusion inhibitor. Nat. Commun 5, 3067 (2014)), and its titer was quantitated by using HIV-1 p24 ELISA. The pseudovirus is then used to infect target Huh-7 cells (or ACE2/293 T cells for pseudo-typed SARS-CoV) (10⁴ per well in 96-well plates) in the presence or absence of serially diluted test mAb. Twelve hours after infection, culture medium is refreshed and then incubated for an additional 48 hours, followed by washing cells with PBS, lysing cells with lysis reagent (Promega), and transferring the cell lysates to 96-well Costar flat-bottom luminometer plates (Corning Costar) for the detection of relative light units using the Firefly Luciferase Assay Kit (Promega) and an Ultra 384 luminometer (Tecan).

3. Inhibition of Live HCoV's Replication

The inhibitory activity of mAbs against OC43 replication in HCT-8 cells is assessed, as described elsewhere (E. Brison, H. Jacomy, M. Desforges, P. J. Talbot, Novel treatment with neuroprotective and antiviral properties against a neuroinvasive human respiratory virus. J. Virol. 88, 1548-1563 (2014)). Briefly, 100 TCID50 of OC43 is mixed with a serial dilution of antibody and incubated at 37° C. for 30 min. The mixture is then applied in triplicate onto the monolayer of HCT-8 cells grown in a 96-well microtiter plate. On day 5 after infection, viral titer in the culture medium is tested, and TCID50 was calculated on the basis of the cytopathic effect (CPE) (J. Ciejka, K. Wolski, M. Nowakowska, K. Pyrc, K. Szczubialka, Biopolymeric nano/microspheres for selective and reversible adsorption of coronaviruses. Mater. Sci. Eng. C Mater. Biol. Appl. 76, 735-742 (2017)). The inhibitory activity of the tested antibodies against 229E replication in A549 cells and NL63 replication in LLC-MK2 cells is evaluated in a similar way, as described above.

The inhibitory activity of antibody against MERS-CoV replication is tested in Calu-3 cells using a modified standard microneutralization assay, as previously described (X. Tao, F. Mei, A. Agrawal, C. J. Peters, T. G. Ksiazek, X. Cheng, C.-T. K. Tseng, Blocking of exchange proteins directly activated by cAMP leads to reduced replication of Middle East respiratory syndrome coronavirus. J. Virol. 88, 3902-3910 (2014)). Briefly, 60 μl of a serially twofold diluted mAb is incubated with 60 μl (120 TCID50) of MERS-CoV in MEM medium supplemented with 2% FBS (M-2 medium) in duplicate wells of 96-well plates for ˜60 min at room temperature. One hundred microliters of the mAb/MERS-CoV mixtures is then transferred into confluent Calu-3 cells grown in 96-well plates. Wells of Calu-3 cells cultured with M-2 medium with and without virus were included in these assays as positive and negative controls, respectively. Supernatants are harvested at 72 hours and infectious virus titers quantified by the standard Vero E6-based infectivity assays and expressed the titers as log 10 TCID50/ml.

### Example 6

Antibodies from Example 5 that are immunoreactive to at least one common human coronavirus selected from HCoV-NL63, HCoV-OC43, HCoV-229E and HCoV-HKU1 and at least one highly pathogenic human coronavirus selected from SARS-CoV-1, MERS-CoV and SARS-CoV-2 are selected for in vivo efficacy studies. In vivo virus challenge studies are performed as follows.

SARS-CoV-2 and SARS-CoV

Monoclonal antibodies (0.1-2 mg/kg) are administered intranasally to golden Syrian Hamsters. After 2 hours the golden Syrian Hamsters are challenged with SARS-CoV-2 or SARS-CoV as described previously (Sia, S. F., Yan, L., Chin, A. W. H. et al. Pathogenesis and transmission of SARS-CoV-2 in golden hamsters. Nature (2020). Weight of the SARS-CoV-2 infected animals is measured from pre-infection until 14 days after infection. Antibodies that provide a protective effect in vivo will prevent or reduce weight loss as compared to untreated animals.

To determine the effect of monoclonal antibodies in golden Syrian Hamsters infected with SARS-CoV, lung pathology is scored eight days after infection. Antibodies that provide a protective effect in vivo will prevent or reduce lung pathology as compared to untreated animals

MERS-CoV

Monoclonal antibodies (0.1-2 mg/kg) are administered intranasally to hDPP4-transgenic mice. After 2 hours the hDPP4-transgenic mice are challenged with MERS-CoV as described previously (Kim J et al. Middle East Respiratory Syndrome-Coronavirus Infection Into Established hDPP4-Transgenic Mice Accelerates Lung Damage Via Activation of the Pro-Inflammatory Response and Pulmonary Fibrosis. J Microbiol Biotechnol. 2020 Mar. 28; 30(3):427-438). Weight of the MERS-CoV infected mice is measured from pre-infection until 14 days after infection. Antibodies that provide a protective effect in vivo will prevent or reduce weight loss as compared to untreated animals.

### Example 7

Venous blood samples were collected from adult volunteers (ranging in age between 40-62 years old) living in Leiden, the Netherlands. PBMC were isolated and cryopreserved for later analysis. All participants answered not to have been in contact with persons (possibly) infected with coronavirus in the 10 days prior to blood collection, and none had previously tested positive for SARS-CoV-2. Nasal swabs were collected and tested in a multiplex respiratory virus PCR including amongst other human coronaviruses NL63, OC43, HKU1, 229E and SARS-CoV-2: all volunteers tested negative for all viruses. Serum samples were diluted 1:4,000 for assessing IgG antibody titers against the Spike protein of hCoVs NL63, OC43, HKU1, and 229E, and diluted 1:200 for assessing IgG antibody titers against the Spike protein of pathogenic hCoVs SARS-COV-1, MERS-COV and SARS-CoV-2 in a multiplex Luminex assay. Based on previous findings (Grobben et al. 2021), a donor was considered to be positive for prior infection(s) if returning a result of ≥125 MFI for IgG against NL63, OC43, HKU1, and 229E, and a result of ≥2,500 MFI for IgG against SARS-CoV-1, MERS-CoV and SARS-CoV-2. Based on these cut-offs each volunteer was considered to have been infected in the past with all four hCoVs NL63, OC43, HKU1, and 229E (Donor ID Nos:1001-1020; Table 1); all tested negative for prior infections with MERS-CoV and SARS-CoV-1, and all except for 2 tested negative for prior SARS-CoV-2 infection (Donor ID Nos: 1007 and 1017 tested positive and are not included in Table 1).

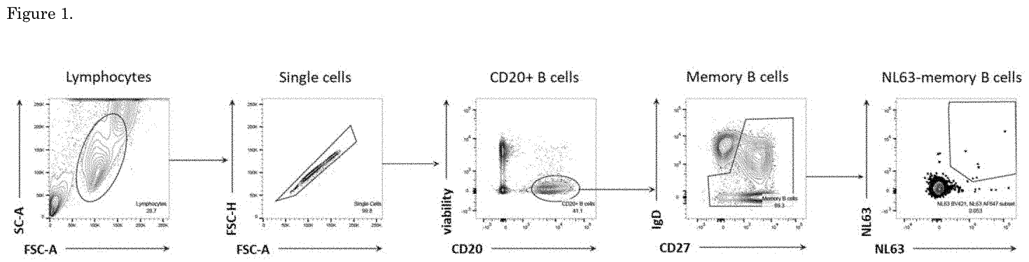

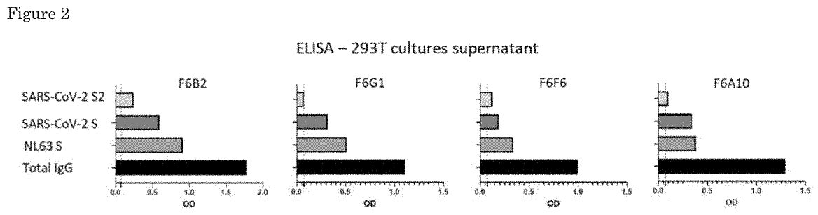

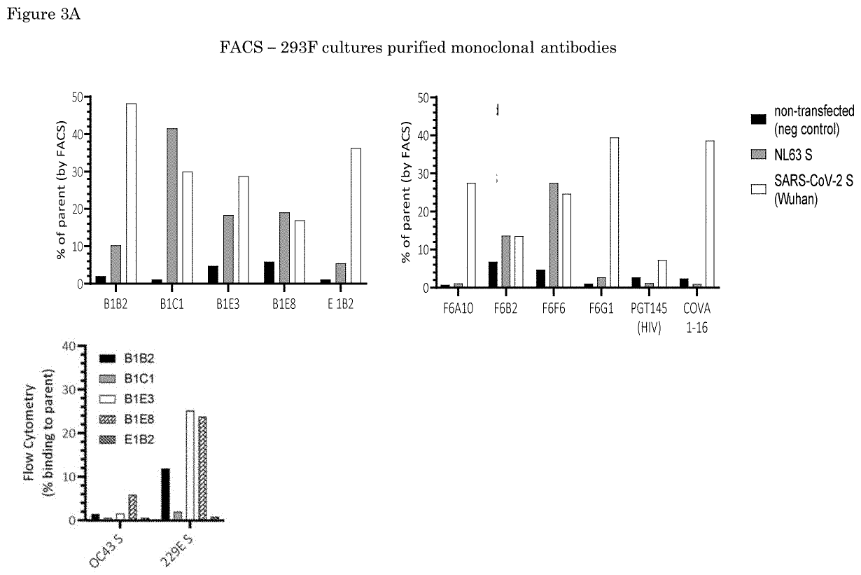

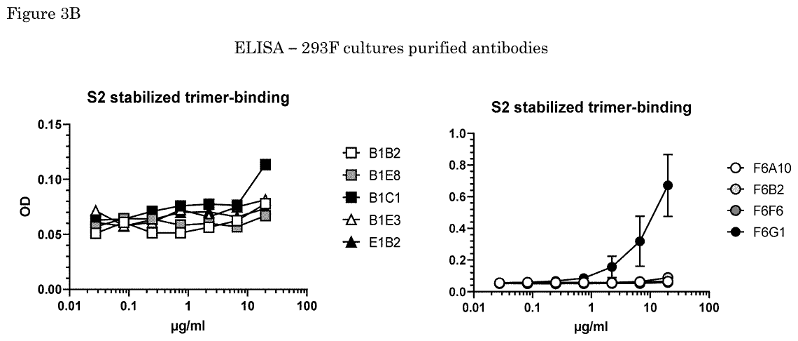

Peripheral blood mononuclear cells (PBMCs) were obtained from venous blood samples collected from human donors and enriched for B-cells. B-cell-enriched PBMCs were single-cell sorted in FACS based on memory B-cell markers and binding to common hCOV NL63 Spike (S) protein, as illustrated in FIG. 1. mRNA was obtained from NL63 S-specific single-sorted B-cells and transcribed to cDNA, and the V(D)J variable regions of the antibodies expressed by the B cells were amplified by PCR. Table 1 shows as an example the number of NL63 S-specific B-cells that could be single-cell sorted and number of successful VH VL pairs that could derived from different donors. The table also shows how many of the sorted NL63-S specific memory B-cells and VH VL pairs derived hereof were binding to SARS-CoV-2 S in the FACS assay pre-sorting as well as the isotype of antibody expressed. For NL63 S memory B-cells single-cell sorted in sorts B, E and F, the variable V(D)J-region of the heavy and light chain of the recombinant antibodies were then cloned into expression vectors containing the constant regions of the human IgG1 for the heavy or light chain using Gibson Assembly. Adherent HEK293T cells were used for small-scale transfection, and supernatants harvested 48 hours post-transfection. Different methods were used to analyse the supernatants, including a Luminex or ELISA assay assessing binding of antibodies to Spike protein of common hCoV NL63 and pathogenic hCoV SAR-CoV-2 (Wuhan), and a FACS analysis for assessing binding of antibodies to Spike protein of common hCoVs NL63 and OC43 and two variants of the pathogenic hCoV SARS-CoV-2 (Brasil and South Africa variants) (due to limited volume available for analysis, supernatants derived from different sorts were analysed testing different methods). Results are shown in FIG. 2 and Tables 2 and 3: multiple clones were identified that produced antibodies binding to the S protein of at least one common hCoV and the S protein of pathogenic hCoV SARS-CoV-2, with some clones in addition showing binding to a stabilized SARS-CoV-2 S2 trimer. These clones were subsequently selected for transfection and larger-scale expression of mAbs in suspension HEK293F cells. Purified antibodies were tested in a FACS analysis for binding to Spike protein of common hCoV NL63 and pathogen hCoV SARS-CoV-2, and in an ELISA assay for binding to a stabilized trimer of the S2 subunit of SARS-CoV-2. Results are shown in FIGS. 3A and 3B and Table 4.

TABLE 1

| | | | | | | | Successful | |
|---|---|---|---|---|---|---|---|---|
| | | NL63 S-binding | | | | | VH VL Pairs | |
| | | memory B-cells sorted | | | | | | From NL63 S- |
| | | | SARS-CoV2 S | Isotype NL63 S- | | | Total from | sorted cells |
| | | | cross-binding | sorted cells | | | NL63 S | cross-binding |
| Donor | Sort | NL63-S | in FACS | IgM | IgG | IgA | sorted cells | SARS-CoV2-S |
| ID_1001 | C | 24 | 3 | 2 | | 1 | 11 | 1 |
| ID_1002 | B | 47 | 2 | — | 2 | — | 20 | 0 |
| ID_1002 | E | 143 | 18 | 40 | 64 | 0 | 81 | 12 |
| ID_1003 | E | 49 | 7 | 16 | 20 | 0 | 35 | 5 |
| ID_1004 | B | 2 | 0 | — | — | — | 0 | 0 |
| ID_1005 | B | 67 | 7 | 4 | 1 | 2 | 24 | 3 |
| ID_1005 | D | 103 | 15 | 66 | 31 | 3 | 25 | 3 |
| ID_1006 | D | 29 | 3 | 16 | 8 | 3 | 8 | 2 |
| ID_1008 | D | 66 | 4 | 31 | 22 | 11 | 22 | 3 |
| ID_1009 | D | 90 | 0 | 72 | 7 | 11 | 10 | 0 |
| ID_1011 | C | 72 | 18 | 8 | 8 | 2 | 41 | 11 |
| ID_1011 | E | 127 | 19 | 43 | 52 | 2 | 90 | 14 |
| ID_1012 | F | 93 | 6 | 62 | 18 | 9 | 68 | 4 |
| ID_1013 | C | 73 | 5 | 4 | 1 | | 25 | 2 |
| ID_1014 | F | 95 | 19 | 48 | 27 | 8 | 21 | 8 |
| ID_1015 | B | 109 | 6 | 6 | — | — | 41 | 1 |
| ID_1015 | C | 23 | 2 | 1 | | 1 | 5 | 0 |
| ID_1018 | F | 74 | 16 | 33 | 16 | 13 | 60 | 9 |
| ID_1019 | F | 165 | 15 | 113 | 13 | 13 | 84 | 5 |
| ID_1020 | E | 137 | 45 | 44 | 49 | 2 | 77 | 26 |
| ID_1020 | F | 96 | 41 | 24 | 42 | 22 | 54 | 20 |

HEK293T Cultures

HEK293T Cultures Supernatant B/E Sort

Clones derived from ‘sorts B and E’ were transfected in 293T cells and supernatants were tested 1. in a Luminex assay for binding to Spike protein of common hCoV NL63 and pathogenic hCoV SAR-CoV-2 (Wuhan), and 2. in a FACS analysis for binding to Spike protein of common hCoVs NL63 and OC43 and two variants of the pathogenic hCoV SARS-CoV-2 (Brasil and South Africa variants). Data are shown in Table 2 for clones producing antibodies binding to S of at least 1 common hCoV and at least 1 pathogenic SARS-CoV-2 variants: these clones were subsequently selected for transfection in 293F cells.

Three clones (B1C1, B1E3 & E1B2) producing antibodies binding to spike protein of at least 1 common hCoVs (NL63) and at least 1 pathogenic hCoV (B1C1 & B1E3: SARS-CoV-2 Wuhan and Brasil variant; and E1B2: SARS-CoV-2 Wuhan and South African variant). Two clones (B1B2 & B1E8) producing antibodies binding to spike protein of at least 2 common hCoVs (NL63 & OC43) and at least 1 pathogenic hCoV (SARS-CoV-2 Brasil variant).

TABLE 2

| | E1B2 | B1B2 | B1C1 | B1E3 | B1E8 |
|---|---|---|---|---|---|
| LUMINEX | | | | | |
| NL63 S | + | − | ++ | + | + |
| SARS-CoV-2 S | − | − | ++ | − | − |
| FACS | | | | | |
| NL63 S | − | + | ++ | ++ | + |
| OC43 S | − | + | − | − | + |
| SARS-COV-2 (Brasil) S | − | + | ++ | + | ++ |
| SARS-COV-2 (South | + | − | − | − | − |
| Africa) S | | | | | |

HEK293T Cultures Supernatant F Sort

Clones derived from ‘sort F’ were transfected in 293T cells and supernatants were tested in ELISA assays for binding to Spike protein of common hCoV NL63 and pathogenic hCoV SAR-CoV-2 (Wuhan), as well as to a stabilized trimer of the S2 subunit of SAR-CoV-2 (Wuhan). Data are shown for clones producing antibodies binding to NL63 S and SARS-CoV-2 S and that were subsequently selected for transfection in 293F cells (FIG. 2 and Table 3). Four clones (F6B2, F6G1, F6F6 and F6A10) produced antibodies binding to the S protein of common hCoVs NL63 (only common hCoV S tested) and pathogenic hCoV SARS-CoV-2 (only pathogenic hCoV S tested). Clones F6B2 and F6F6 in addition showed binding to the SARS-CoV-2 S2 trimer.

TABLE 3

| ELISA | F6B2 | F6G1 | F6F6 | F6A10 |
|---|---|---|---|---|
| NL63 S | + | + | + | + |
| SARS-CoV-2 S | + | + | + | + |
| SARS-CoV-2 S2-trimer | + | − | + | − |

HEK293F Cultures 293F Culture Purified Antibodies B/E and F Sorts

Clones selected for producing antibodies binding to S of at least 1 common hCoV and pathogenic SARS-CoV-2 were transfected into 293F cells, and antibodies were purified from cultures and tested 1. in a FACS analysis for binding to Spike protein of common hCoV NL63, and hCoVs OC43 and 229E (clones derived from sorts B/E only) and pathogen hCoV SARS-CoV-2 (Wuhan), and 2. in an ELISA assay for binding to a stabilized trimer of the S2 subunit of SARS-CoV-2. Purified antibodies from 6 clones (B1B2, B1C1, B1E3, B1E8, F6B2 & F6F6) showed binding to Spike protein of common hCoV NL63 (for the F sort-derived clones the only common hCoV tested) including 2 clones from the B/E sort binding to the Spike protein of a 2 common hCoV (229E) and 1 clone from this sort (B1E8) binding to Spike protein of 3 tested common hCoVs (NL63, 229E, and OC43), with all 6 clones also binding to pathogenic hCoV SARS-CoV-2 (only Wuhan tested) in FACS. Antibodies produced by clones E1B2, F6A10, and F6G1 showed good binding to SARS-CoV-2 S but low/no binding to NL63 S in the FACS experiment despite parent memory B-cells having been sorted based on their binding to for NL63 S and antibodies produced in the 293T cultures showing good binding to NL63 S. See Table 4 and FIG. 3A.

293F cultures purified antibodies from clone F6G1 showed binding to a stabilized trimer of the S2 subunit of SARS-CoV-2 in ELISA, while there was some evidence that antibodies produced by clone B1C1 bound S2 when tested at higher concentrations. See FIG. 3B and table 4.

TABLE 4A

| | B1B2 | B1C1 | B1E3 | B1E8 | E1B2 |
|---|---|---|---|---|---|
| FACS | | | | | |
| NL63 S | + | ++ | + | + | −/+ |
| OC43 S | − | − | − | + | − |
| 229E S | ++ | − | ++ | ++ | − |
| SARS-CoV-2 S | ++ | + | + | + | + |
| ELISA | | | | | |
| SARS-CoV-2 | − | −/+ | − | − | − |
| S2-trimer | | | | | |

TABLE 4B

| | F6B2 | F6G1 | F6F6 | F6A10 |
|---|---|---|---|---|
| FACS | | | | |
| NL63 S | + | −/+ | + | − |
| OC43 S | ND | ND | ND | ND |
| 229E S | ND | ND | ND | ND |
| SARS-CoV-2 S | + | ++ | + | + |
| ELISA | | | | |
| SARS-CoV-2 | − | + | − | − |
| S2-trimer | | | | |

Materials and Methods

Coronavirus S Protein Designs

A prefusion S protein ectodomain of SARS-CoV-2 with a T4 trimerization domain and hexahistidine (His) tag, and the RBD domain of SARS-CoV-2 were designed and cloned as previously described (Brouwer et al. 2020), and a SARS-CoV-2 S2 ectodomain with a T4 trimerization domain and StrepII-tag was made. Prefusion S protein ectodomains of the other human coronaviruses were designed using this sequence as template and ordered at Genscript. The truncation site was selected by alignment of the different protein sequences. If present, the furin cleavage site was replaced with “GGGG” at amino acids corresponding to 682-685 in the SARS-CoV-2 S protein reference sequence and proline substitutions were inserted at amino acids corresponding to 986 and 987 in the SARS-CoV-2 reference sequence. Genbank ID MN908947.3 (SARS-CoV-2) ABD72984.1 (SARS-CoV), AHI48550.1 (MERS-CoV), AAT84362.1 (OC43-CoV), Q0ZME7 (HKU1-CoV), NP_073551.1 (229E-CoV) and AKT07952.1 (NL63-CoV) served as templates for the protein designs. Avi-tags were added between the trimerization domain and the his-tag for proteins used in flow cytometry.

Expression and Purification of Coronavirus S Proteins

SARS-CoV-1, MERS-CoV, SARS-CoV-2, NL63-CoV, OC43-CoV, HKU1-CoV, and 229E-CoV Spike proteins were produced in HEK293F cells (Invitrogen) maintained in Freestyle medium (Life Technologies). Transfections were performed using Polyethylenimine Hydrochloride (PEI) MAX (Polysciences) at 1 mg/L and the expression plasmids at 312.5 μg/L in a 3:1 ratio in 50 mL OptiMEM (Gibco) per Liter. Supernatants were harvested 7 days post transfection by centrifugation at 4000 rpm for 30 minutes followed by filtration of the supernatant using 0.22 μM Steritop filter units (Merck Millipore). His-tagged proteins were purified from the clarified supernatant with affinity chromatography using Nickel-Nitrilotriacetic Acid (Ni-NTA) agarose beads (Qiagen). Eluates were concentrated and buffer exchanged to PBS using 100 kDa molecular weight cut-off (MWCO) Vivaspin centrifugal concentrators. Further purification to remove aggregated and monomeric protein fractions was performed using Size Exclusion Chromatography on a Superose 6 increase 10/300 GL column (GE Healthcare) using PBS as a buffer. Trimeric S proteins were eluted at a volume of approximately 13 mL. Fractions containing trimeric protein were pooled and concentrated using 100 kDa MWCO Vivaspin centrifugal concentrators. Resulting protein concentrations were determined using a Nanodrop 2000 Spectrophotometer. Proteins were stored at −80° C. until needed.

Proteins with avi-tags were biotinylated with the BirA kit (Avidity) using the same conditions for all proteins according to manufactures protocol. Subsequently, proteins were further purified by Size Exclusion Chromatography (SEC), using a SuperDex200 10/300 GL increase column. The peak-fractions corresponding to the S timer protein were pooled, concentrated again, and stored in PBS at −80° C.

Luminex Assay Assessing Antibody Binding to Coronavirus S Proteins

Proteins were covalently coupled to Luminex Magplex beads using a two-step carbodiimide reaction. SARS-CoV-2 S protein was coupled at a ratio of 75 μg protein to 12.5 million beads. Other proteins were coupled equimolar to SARS-CoV-2 S protein. SARS-CoV-2 S1 and S2 proteins were obtained from Abclonal Luminex Magplex beads (Luminex) were washed with 100 mM monobasic sodium phosphate pH 6.2 and activated by addition of Sulfo-N-Hydroxysulfosuccinimide (Thermo Fisher Scientific) and 1-Ethyl-3-(3-dimethylaminopropyl) carbodiimide (Thermo Fisher Scientific) and incubated for 30 minutes on a rotator at room temperature. The activated beads were washed three times with 50 mM MES pH 5.0. Proteins were diluted in 50 mM MES pH 5.0 and added to the beads. The beads and proteins were incubated for three hours on a rotator at room temperature. Next, the protein-conjugated beads were washed with PBS and blocked with PBS containing 2% BSA, 3% Fetal calf serum (FCS) and 0.02% Tween-20 at pH 7.0 for 30 minutes on a rotator at room temperature. Protein-conjugated beads were washed and stored at 4° C. in PBS containing 0.05% Sodium Azide and used within 6 months. Detection of the His-tag on each S protein-coupled bead was used to confirm the amount of protein on the beads.

50 μl of a working bead mixture containing 20 beads per μ1 of each protein-bead conjugate was incubated overnight with 50 μl of diluted serum. Plates were sealed and incubated on a plate shaker overnight at 4° C. The next day, plates were washed with TBS containing 0.05% Tween-20 (TBST) using a hand-held magnetic separator. Protein-conjugated beads were resuspended in 50 μl of Goat-anti-human IgG-PE (Southern Biotech) and incubated on a plate shaker at room temperature for 2 hours. Next, beads were washed with TBST and resuspended in 70 μl Magpix drive fluid (Luminex) and left for a few minutes on a plate shaker at room temperature. Read-outs were then performed on a Magpix (Luminex). Median fluorescence intensity (MFI) values were assessed as the median of approximately 50 beads per well and were corrected by subtraction of MFI values from buffer and beads only wells.

Single-Cell Memory-B Cell Sorting

To identify B-cells binding to coronavirus proteins in flowcytometry, biotinylated recombinant CoV (NL63) and SARS-CoV-2 Spike (S) proteins were conjugated with a streptavidin fluorophore resulting in fluorescent labelled-probes, as described in Brouwer et al. (Science 2020). Briefly, recombinant NL63 S protein was conjugated in a 2:1 molar ratio to streptavidin-conjugates AF647 (0.5 mg/mL, BioLegend) and BV421 (0.1 mg/mL BioLegend). Recombinant SARS-CoV-2 S protein was labeled with streptavidin BB515 (0.1 mg/mL BD Biosciences). The conjugation incubation took place at 4° C., for a minimum of 1 hour. Probe conjugation was stopped by a 15 min incubation with 10 mM free biotin (Genecopoeia).

Peripheral blood mononuclear cells (PBMCs) were first enriched for B-cells using a Human B cell enrichment kit (Stemcell) according to manufacturer's instructions. B-cell enriched PBMCs were then incubated for 30 min at 4° C. with the fluorescent labelled coronavirus) Spike proteins (NL63S-AF647; NL63S-BV421; SARS-CoV-2 S-BB515; a live/dead-cell marker (viability-eF780, eBiosciences); the surface markers CD20-PE-CF594 (2H7, BD Biosciences), CD27-PE (L128, BD Biosciences), IgG AF700 (G18-145, BD Biosciences), IgM-BV605 (MHM-88, BioLegend), IgD-PE-Cy7 (IA6-2, Biolegend); and various surface markers with the same fluorophore APC-eF780 to eliminate all non-B cells, including T-cell markers CD3 (UCHT1, eBiosciences) and CD4 (OKT4, eBiosciences), monocyte and macrophage marker CD14 (C1D3, eBiosciences), and NK-cell marker CD16 (CB16, eBiosciences). Following three washes in PBS (Dulbecco's Phosphate-Buffered Saline, eBiosciences) supplemented with 1 mM EDTA and 2% fetal calf serum, flow cytometry was performed on a 4-laser FACS ARIA (BD Biosciences). Live memory B-cells were analysed for binding of NL63 S (AF647 and BV421), SARS-CoV-2 S, and isotype expression using FlowJo (version 10.6.2). Live memory B cells that were double positive for NL63 S (AF647 and BV421) (see example FIG. 1) were single cell sorted using yield purity into empty 96-well plates and immediately frozen at −80° C. for at least 1 h before lysing and performing reverse transcriptase (RT)-PCR to transcribe the mRNA to cDNA.

Extraction of Antibody cDNA from Memory B-Cells

Frozen single-cell sorted memory B-cells were reconstituted in lysis buffer (at room temperature), consisting of 20 U Ribonuclease (RNAse) inhibitor (Invitrogen), first strand SuperScript III buffer (Invitrogen), and 1.25 μl of 0.1M Dithiothreitol (DTT) (Invitrogen) in a total volume of 20 μl. mRNA of the lysed NL63 S protein specific single B cells was converted into cDNA by RT-PCR. Briefly, 50 U SuperScript III RTase (Invitrogen), 2 μl of 6 mM dNTPs (Invitrogen), and 200 ng random hexamer primers (Thermo Scientific) in a total volume of 6 μl was added to each well containing a single lysed cell. The following RT program was used: 10 min at 42° C., 10 min 25° C., 60 min at 50° C., 5 min at 95° C., and infinity 4° C. cDNA was stored at −20° C. until further analysis.

The V(D)J variable regions of the antibodies expressed by the NL63 S-specific single cell sorted B cells were amplified as previously described by Tiller et al J Immunol Methods 2008. Briefly, for both the kappa and lambda chain, PCR 1 was performed with 0.5 U MyTaq polymerase (BioLine), 0.1 μM of both forward and reverse multiplex primers, MyTaq PCR reaction buffer (BioLine), and 2 μl of cDNA, in a total volume of 20 μl for 1 min 95° C., 50 cycles of 15 s at 95° C., 15 s at 58° C., 45 s at 72° C., followed by 10 min at 72° C. The nested PCR was performed with 0.375 U HotStarTaq Plus polymerase (Qiagen), 0.2 mM dNTPs, 0.034 μM of both forward and reverse multiplex primers, HotstarTaq Plus PCR buffer (Qiagen), and 2 μl of PCR 1 product in a total volume of 14.5 μl for 5 min at 95° C., 50 cycles of 30 s at 94° C., 30 s at 60° C., 1 min at 72° C., followed by 10 min at 72° C. For the heavy chain a primary and two nested PCR reactions were performed. Briefly, the primary PCR was performed with 0.375 U HotStarTaq Plus polymerase (Qiagen), 0.2 mM dNTPs, 0.069 μM of both forward and reverse multiplex primers, HotstarTaq Plus PCR buffer (Qiagen), and 2 μl of cDNA in a total volume of 14.5 μl for 5 min at 95° C., 50 cycles of 30 s at 94° C., 30 c at 52° C., 1 min at 72° C., followed by 10 min at 72° C. The first nested PCR was performed with 0.5 U MyTaq polymerase (Bioline), 0.05 μM of both forward and reverse multiplex primers (39), MyTaq PCR reaction buffer (BioLine), and 2 μl of PCR 1 product in a total volume of 20 μl for 1 min 95° C., 30 cycles of 15 s at 95° C., 15 s at 58° C., 45 s at 72° C., followed by 10 min at 72° C. The final PCR, was performed with 0.375 U HotStarTaq Plus polymerase (Qiagen), 0.2 mM dNTPs, 0.034 μM of both forward and reverse multiplex primers with vector overhang, HotstarTaq Plus PCR buffer (Qiagen), and 2 μl of PCR 2 product in a total volume of 14.5 μL for 5 min at 95° C., 50 cycles of 30 s at 94° C., 30 s at 60° C., 1 min at 72° C., followed by 10 minutes at 72° C.

Antibody Cloning and Small-Scale Expression

All recombinant antibodies were expressed in a mammalian cell expression system as described previously by Sok et al. (PNAS 2014) and van Gils et al. (Nat Microbiol 2016). Briefly, the variable V(D)J-region of the heavy and light chain of the antibody were cloned into corresponding expression vectors containing the constant regions of the human IgG1 for the heavy or light chain using Gibson Assembly (Gibson et al. Nat Methods 2009). The Gibson Assembly was carried out with a home-made Gibson mix consisting of 2× Gibson mix (0.2 U T5 exonuclease (Epibio), 12.5 U Phusion polymerase (New England Biolabs), Gibson reaction buffer (0.5 g PEG-8000 (Sigma Life Sciences), 1 M Tris/HCl pH 7.5, 1 M MgCl2, 1 M DTT, 100 mM dNTPs, 50 mM NAD (New England Biolabs), MQ)) and performed for 60 min at 50° C. The sequence integrity of the plasmids was verified by Sanger sequencing. For small-scale transfection, adherent HEK293T cells (ATCC, CRL-11268) were maintained in Dulbecco's Modified Eagle's Medium (DMEM) supplemented with 10% fetal calf serum (FCS), penicillin (100 U/mL), and streptomycin (100 μg/mL) and transfected as described previously by van Gils et al. (Nat Microbiol 2016). HEK293T cells were seeded 24 h prior to transfection in 24-well or 48-well plates at a density of 2.75×10\hat{ }5 or 1.5×10\hat{ }5 cells per well, respectively, in complete medium as described above. The transfection mix consisted of a 1:1 (w:w) HC/LC ratio using a 1:2.5 ratio with lipofectamin 200 (Invitrogen) in 200 or 100 μL Opti-MEM, respectively. After 15 minutes of incubation at room temperature, the transfection mix was added to the cells. Supernatants were harvested 48 h post-transfection, clarified and stored at 4° C. until further analyses.

Larger-Scale Antibody Expression and Purification

For larger-scale expression of selected mAbs, suspension HEK293F cells (Invitrogen, cat no. R79007) were cultured in FreeStyle medium (Gibco) and co-transfected with the two IgG plasmids expressing the corresponding heavy chain and light chain in a 1:1 ratio at a density of 0.8-1.2 million cells/mL in a 1:3 ratio with 1 mg/L PEImax (Polysciences). The recombinant IgG antibodies were isolated from the cell supernatant after five days of culture as described previously (Sok et al. PNAS 2014). In short, the cell suspension was centrifuged 25 min at 4000 rpm, and the supernatant was filtered using 0.22 μm pore size SteriTop filters (Millipore). The filtered supernatant was run over a 10 mL protein A/G column (Pierce) followed by two column volumes of PBS wash. The antibodies were eluted with 0.1 M glycine pH 2.5, into the neutralization buffer 1 M TRIS pH 8.7 in a 1:9 ratio.

The purified antibodies were buffer exchanged to PBS using 100 kDa VivaSpin20 columns (Sartorius). The IgG concentration was determined on the NanoDrop 2000 and the antibodies were stored at 4° C. until further analyses.

Ni²⁺-nitrilotriacetic Acid (Ni-NTA)-Capture ELISA

His-tagged Spike proteins and StrepII-tagged S2 protein of SARS-CoV-2 were loaded in casein (Thermo Scientific) on 96-well Ni-NTA plates (Qiagen) for 2 h at RT. After the plates were washed with Tris Buffered Saline (TBS), three-fold serial dilutions of mAbs in casein, starting from a 10 μg/mL concentration, or HEK 293T antibody transfected supernatant were added. Following three washes with TBS, a 1:3000 dilution of HRP-labeled goat anti-human IgG (Jackson Immunoresearch) in casein was added for 1 h at RT. Finally, after washing the plates five times with TBS/0.05% Tween-20, developing solution (1% 3,3′,5,5′-tetramethylbenzidine (Sigma-Aldrich), 0.01% hydrogen peroxide, 100 mM sodium acetate and 100 mM citric acid) was added. Development of the colorimetric endpoint proceeded for 4 min before termination by adding 0.8 M sulfuric acid and optical density (OD value) were determined at 450 nm.

Full Length SARS-CoV-2 and CoV Spike Expression and Binding in FACS

Surface expressed SARS-CoV-2 and CoV spikes were obtained by transfection of 8 μg of SARS-CoV-2 full length plasmid DNA and 25 μl PEImax in 400 μl Optimem onto 12 to 15 mL HEK293T cells in a petri-dish (seeded the day before 3.0×10\hat{ }6). After 48 h cells were harvested and frozen. After thawing, 293T cells expressing the Spike protein of interest were plated at 20,000 to 30,000 cells in PBS/0.5% FCS (FACS buffer) per well in a 96-well plate and incubated 1:1 with unpurified sup from 293T cells, or with a dilution of mAb after purification and production in 293F cells for 1 h at 4° C. Cells were subsequently washed twice with FACS buffer and stained, for 30 min at 4° C. and in the dark, in 50 μl FACS buffer containing 1:1000 diluted PE-conjugated goat F(ab)′2 anti-human IgG (Southern Biotech 2042-09). Cells were washed once more with FACS buffer and analyzed on the FACS canto II analyzer (BD). Samples were analyzed by FlowJo software and percentage of cells that show binding were plotted.

Methods are Further Described in the Following References.
Tiller, T, Meffre, E, Yurasov, S, Tsuiji, M, Nussenzweig, M C, Wardemann, H. Efficient generation of monoclonal antibodies from single human B cells by single cell RT-PCR and expression vector cloning. J Immunol Methods 2008; 329: 112-124.
Gibson, D G, Young, L, Chuang, R-Y, Venter, J C, Hutchison, C A, Smith, H O. Enzymatic assembly of DNA molecules up to several hundred kilobases. Nat Meth 2009; 6: 343-345.
Sok, D, van Gils, M J, Pauthner, M, Julien, J-P, Saye-Francisco, K L, Hsueh, J, Briney, B, et al. Recombinant HIV envelope trimer selects for quaternary-dependent antibodies targeting the trimer apex. Proc Natl Acad Sci USA 2014; 111: 17624-17629.
van Gils, M J, van den Kerkhof, T L G M, Ozorowski, G, Cottrell, C A, Sok, D, Pauthner, M, Pallesen, J, et al. An HIV-1 antibody from an elite neutralizer implicates the fusion peptide as a site of vulnerability. Nature Microbiology 2016; 2: 16199.
Brouwer, P J M, Caniels, T G, van der Straten, K, Snitselaar, J L, Aldon, Y, Bangaru, S, Tones, J L, et al. Potent neutralizing antibodies from COVID-19 patients define multiple targets of vulnerability. Science 2020; 38: eabc5902.
Marloes Grobben, et al. Cross-reactive antibodies after SARS-CoV-2 infection and vaccination. medRxiv 2021. doi: https://doi.org/10.1101/2021.05.26.21256092
van Haaren, M M, McCoy, L E, Tones, J L, Lee, W, Cottrell, C A, Copps, J L, van der Woude, P, et al. Antibodies from rabbits immunized with HIV-1 clade B SOSIP trimers can neutralize multiple clade B viruses by destabilizing the envelope glycoprotein. J Virol 2021; JVI0009421. Jelle van Schooten, et al. Antibody responses induced by SHIV infection are more focused than those induced by soluble native HIV-1 envelope trimers in non-human primates. Plos Pathogens 2021.

1. A method for identifying a coronavirus cross-reacting antibody, said method comprising:
a) providing plasma samples from one or more human subjects said samples collected, independently, at a time point (X),
b) identifying subjects having plasma samples with immunoglobulins that bind to at least two human coronaviruses (HCoV), wherein the HCoV is selected from HCoV-NL63, HCoV-OC43, HCoV-229E and HCoV-HKU1;
c) providing PBMC samples from said identified subjects, wherein the PBMC samples are collected at time point (X) or later and comprise B-cells selected from memory B-cells, plasma cells, and plasmablasts;
d) screening antibodies, or antigen-binding fragments thereof, encoded by the B-cells of c) for binding to at least part of the S2 ectodomain of the S (spike) protein from at least two different coronaviruses;
e) selecting antibodies, or antigen-binding fragments thereof, that bind to at least part of the S2 ectodomain of the S protein of at least one common human coronavirus selected from HCoV-NL63, HCoV-OC43, HCoV-229E and HCoV-HKU1 and that bind to at least part of the S2 ectodomain of the S protein of at least one highly pathogenic human coronavirus selected from SARS-CoV-1, MERS-CoV and SARS-CoV-2;
f) selecting antibodies or antigen-binding fragments thereof from e) that inhibit viral fusion, infection, and/or replication of at least one common human coronavirus selected from HCoV-NL63, HCoV-OC43, HCoV-229E and HCoV-HKU1 and that inhibit viral fusion, infection, and/or replication of at least one highly pathogenic human coronavirus selected from SARS-CoV-1, MERS-CoV and SARS-CoV-2;
g) determining the ability of the selected antibodies, or antigen-binding fragments thereof, from f) to prevent or reduce infection in an in vivo model of HCoV infection selected from SARS-CoV-1, MERS-CoV and SARS-CoV-2; and
h) selecting antibodies, or antigen-binding fragments thereof, that prevent or reduce infection in an in vivo model of HCoV infection selected from SARS-CoV-1, MERS-CoV and SARS-CoV-2.

2. The method of claim 1, further comprising
providing a further plasma sample from a plurality of subjects, wherein said sample is collected at a time point (Y), wherein time point (Y) is at least 3 months earlier or later than time point (X).

3. The method of claim 2, wherein plasma samples from a subject are selected which have an increase in immunoglobulins that bind to at least two HCoV's as compared to plasma samples from the subject collected at an earlier or later time point.

4. The method of claim 1, wherein the plasma samples have IgG, IgM, and/or IgA immunoglobulins that bind, independently, to at least two HCoVs.

5. The method of claim 4, wherein the immunoglobulins bind the S2 domain of a HCoV spike protein.

6. The method of claim 1, wherein step d) comprises screening antibodies, or antigen-binding fragments thereof, for binding to at least part of the fusion peptide, the HR1 heptad repeat, or the HR2 heptad repeat of the S protein from at least two different coronaviruses.

7. The method of claim 1, wherein step e) comprises selecting antibodies, or antigen-binding fragments thereof, that bind to at least part of the S2 domain of the S protein of HCoV-NL63, HCoV-OC43, HCoV-229E and HCoV-HKU1.

8. The method of claim 1, wherein step e) comprises selecting antibodies, or antigen-binding fragments thereof, that bind to at least part of S2 domain of the S protein of SARS-CoV-1, MERS-CoV and SARS-CoV-2.

9. The method of claim 1, wherein step g) comprises determining the ability of the selected antibodies, or antigen-binding fragments thereof, to prevent or reduce infection in an in vivo model of HCoV infection from SARS-CoV-1, MERS-CoV, and SARS-CoV-2.

10. The method of claim 1, wherein step h) comprises selecting antibodies, or antigen-binding fragments thereof, that prevent or reduce infection in an in vivo model of HCoV infection from SARS-CoV-1, MERS-CoV and SARS-CoV-2.

11. The method of claim 1, comprising
selecting antigen-binding fragments that bind to at least part of the S2 domain of at least one common human coronavirus selected from HCoV-NL63, HCoV-OC43, HCoV-229E and HCoV-HKU1 and that bind to at least part of the S2 domain of the S protein of at least one highly pathogenic human coronavirus selected from SARS-CoV-1, MERS-CoV and SARS-CoV 2
preparing IgM, IgA, or IgG antibodies comprising the selected antigen-binding fragments,
determining the ability of the IgM, IgA, or IgG antibodies to prevent or reduce infection in an in vivo model of HCoV infection selected from SARS-CoV-1, MERS-CoV and SARS-CoV-2; and
selecting IgM, IgA, or IgG antibodies that prevent or reduce infection in an in vivo model of HCoV infection selected from SARS-CoV-1, MERS-CoV and SARS-CoV-2.

12. An antibody or antigen binding fragment thereof identified by a method according to claim 1.

13. (canceled)

14. A method of treating or preventing infection by a coronavirus comprising administering locally to a subject in need thereof the antibody of claim 12.

15. (canceled)

16. The method of claim 1, wherein said one or more human subjects are least 45 years or older.

17. The method of claim 1, wherein step b) comprises identifying subjects having plasma samples with immunoglobulins that bind to at least four human coronaviruses (HCoV), wherein the HCoV is selected from HCoV-NL63, HCoV-OC43, HCoV-229E and HCoV-HKU1.

18. The method of claim 1, wherein step d) comprises screening antibodies, or antigen-binding fragments thereof, encoded by the B-cells of c) for binding to at least part of the S2 ectodomain of the S (spike) protein from at least four different coronaviruses.

19. The method of claim 1, wherein said selected antibody or antigen-binding fragment thereof also binds to at least part of the S2 ectodomain of the S protein of an animal coronavirus.

20. The method of claim 1, wherein said selected antibody or antigen-binding fragment thereof also binds to at least part of the S2 ectodomain of the S protein of an animal coronavirus.
